SEQUENCE LISTING (35 sequences)

SEQ ID NO: 1 - protein, 373 aa, Mycobacterium avium
MTKPLTDTAPVDPGAQRGSMPLTNRIQGAVTSVGVKVIPWIPTAVRRGLVRGRSVIIDGNTLDPTLQLMLSGLRAVGIDGLVVDDDPELSRAQMHESTVGFPGPQIHVDVAELALPGPAGDIPARHYRPAGGETQAPLLVFYHGGGWSIGDLDTHDSLCRLTCRDAGIHVLSIDYRLAPEHPAPAAIDDAYAAFTWAHEHAGELGAIPGRVAVGGDSAGGNLAAVVSQLARDAGGPAPVLQWLIYPRTDFTARTRSLSLFSRGFLLTKRDIDWFESQYLRNSRLDRTDPRVSPALAESLAGLAPALIAVAGFDPLRDEGQSYAEALRAAGTPVDLRYLGSLTHGFANLFQLGGDSMVATSELISALRAHLSRV

SEQ ID NO: 2 - protein, 376 aa, Mycobacterium bovis
MTKSLPGVADLRLGANHPRMWTRRVQGTVVNVGVKVLPWIPTPAKRILSAGRSVIIDGNTLDPTLQLMLSTSRIFGVDGLAVDDDIVASRAHMRAICEAMPGPQIHVDVTDLSIPGPAGEIPARHYRPSGGGATPLLVFYHGGGWTLGDLDTHDALCRLTCRDADIQVLSIDYRLAPEHPAPAAVEDAYAAFVWAHEHASDEFGALPGRVAVGGDSAGGNLSAVVCQLARDKARYEGGPTPVLQWLLYPRTDFTAQTRSMGLFGNGFLLTKRDIDWFHTQYLRDSDVDPADPRLSPLLAESLSGLAPALIAVAGFDPLRDEGESYAKALRAAGTAVDLRYLGSLTHGFLNLFQLGGGSAAGTNELISALRAHLSRV

SEQ ID NO: 3 - protein, 378 aa, synthetic construct
  REGION 1..378: note = synthetic consensus sequence
  VARIANT 4: note = X can be any naturally occurring amino acid
  VARIANT 6..8: note = X can be any naturally occurring amino acid
  VARIANT 10..13: note = X can be any naturally occurring amino acid
  VARIANT 16..19: note = X can be any naturally occurring amino acid
  VARIANT 22: note = X can be any naturally occurring amino acid
  VARIANT 24: note = X can be any naturally occurring amino acid
  VARIANT 26: note = X can be any naturally occurring amino acid
  VARIANT 29: note = X can be any naturally occurring amino acid
  VARIANT 31..32: note = X can be any naturally occurring amino acid
  VARIANT 38: note = X can be any naturally occurring amino acid
  VARIANT 44..46: note = X can be any naturally occurring amino acid
  VARIANT 48: note = X can be any naturally occurring amino acid
  VARIANT 50..51: note = X can be any naturally occurring amino acid
  VARIANT 72..73: note = X can be any naturally occurring amino acid
  VARIANT 75..76: note = X can be any naturally occurring amino acid
  VARIANT 78: note = X can be any naturally occurring amino acid
  VARIANT 82: note = X can be any naturally occurring amino acid
  VARIANT 87..89: note = X can be any naturally occurring amino acid
  VARIANT 93: note = X can be any naturally occurring amino acid
  VARIANT 95..101: note = X can be any naturally occurring amino acid
  VARIANT 111..112: note = X can be any naturally occurring amino acid
  VARIANT 114..115: note = X can be any naturally occurring amino acid
  VARIANT 121: note = X can be any naturally occurring amino acid
  VARIANT 130: note = X can be any naturally occurring amino acid
  VARIANT 133..134: note = X can be any naturally occurring amino acid
  VARIANT 136: note = X can be any naturally occurring amino acid
  VARIANT 148..149: note = X can be any naturally occurring amino acid
  VARIANT 157: note = X can be any naturally occurring amino acid
  VARIANT 167: note = X can be any naturally occurring amino acid
  VARIANT 169: note = X can be any naturally occurring amino acid
  VARIANT 187..188: note = X can be any naturally occurring amino acid
  VARIANT 195: note = X can be any naturally occurring amino acid
  VARIANT 202: note = X can be any naturally occurring amino acid
  VARIANT 205: note = X can be any naturally occurring amino acid
  VARIANT 208: note = X can be any naturally occurring amino acid
  VARIANT 224: note = X can be any naturally occurring amino acid
  VARIANT 228: note = X can be any naturally occurring amino acid
  VARIANT 234: note = X can be any naturally occurring amino acid
  VARIANT 242: note = X can be any naturally occurring amino acid
  VARIANT 249: note = X can be any naturally occurring amino acid
  VARIANT 258: note = X can be any naturally occurring amino acid
  VARIANT 262..263: note = X can be any naturally occurring amino acid
  VARIANT 266..267: note = X can be any naturally occurring amino acid
  VARIANT 280..281: note = X can be any naturally occurring amino acid
  VARIANT 286: note = X can be any naturally occurring amino acid
  VARIANT 288..289: note = X can be any naturally occurring amino acid
  VARIANT 291..292: note = X can be any naturally occurring amino acid
  VARIANT 296: note = X can be any naturally occurring amino acid
  VARIANT 299: note = X can be any naturally occurring amino acid
  VARIANT 305: note = X can be any naturally occurring amino acid
  VARIANT 325: note = X can be any naturally occurring amino acid
  VARIANT 329: note = X can be any naturally occurring amino acid
  VARIANT 337: note = X can be any naturally occurring amino acid
  VARIANT 351: note = X can be any naturally occurring amino acid
  VARIANT 359: note = X can be any naturally occurring amino acid
  VARIANT 361..363: note = X can be any naturally occurring amino acid
  VARIANT 365: note = X can be any naturally occurring amino acid
MTKXLXXXAXXXXGAXXXXMPXTXRXQGXVXXVGVKVXPWIPTXXXRXLXXGRSVIIDGNTLDPTLQLMLSXXRXXGXDGLXVDDDXXXSRAXMXXXXXXXPGPQIHVDVXXLXXPGPAGXIPARHYRPXGGXXQXPLLVFYHGGGWXXGDLDTHDXLCRLTCRDAXIXVLSIDYRLAPEHPAPAAXXDAYAAFXWAHEHAXDEXGAXPGRVAVGGDSAGGNLXAVVXQLARDXARYEGGPXPVLQWLXYPRTDFTAXTRSXXLFXXGFLLTKRDIDWFXXQYLRXSXXDXXDPRXSPXLAESLXGLAPALIAVAGFDPLRDEGXSYAXALRAAGTXVDLRYLGSLTHGFXNLFQLGGXSXXXTXELISALRAHLSRV

SEQ ID NO: 4 - DNA, 17 bp, synthetic construct
  misc_feature 1..17: note = synthetic
  source 1..17: mol_type = other DNA
ggtgagcgccagaggaa

SEQ ID NO: 5 - DNA, 21 bp, synthetic construct
  misc_feature 1..21: note = synthetic
  source 1..21: mol_type = other DNA
cagcttctcctcttggtggac

SEQ ID NO: 6 - DNA, 18 bp, synthetic construct
  misc_feature 1..18: note = synthetic
  source 1..18: mol_type = other DNA
aactggatcccagacccc

SEQ ID NO: 7 - DNA, 17 bp, synthetic construct
  misc_feature 1..17: note = synthetic
  source 1..17: mol_type = other DNA
gtagctgctgggagcgc

SEQ ID NO: 8 - DNA, 21 bp, synthetic construct
  misc_feature 1..21: note = synthetic
  source 1..21: mol_type = other DNA
ggaacacgaactccagaaggc

SEQ ID NO: 9 - DNA, 23 bp, synthetic construct
  misc_feature 1..23: note = synthetic
  source 1..23: mol_type = other DNA
acagagttcatgtgatggtccac

SEQ ID NO: 10 - DNA, 17 bp, synthetic construct
  misc_feature 1..17: note = synthetic
  source 1..17: mol_type = other DNA
accaccgtgcgtaccac

SEQ ID NO: 11 - DNA, 20 bp, synthetic construct
  misc_feature 1..20: note = synthetic
  source 1..20: mol_type = other DNA
ggaggtcttgggaccctctc

SEQ ID NO: 12 - DNA, 26 bp, synthetic construct
  misc_feature 1..26: note = synthetic
  source 1..26: mol_type = other DNA
cgttaatagcagttccttctagcaac

SEQ ID NO: 13 - DNA, 22 bp, synthetic construct
  misc_feature 1..22: note = synthetic
  source 1..22: mol_type = other DNA
ggatagccctggatttctgtgc

SEQ ID NO: 14 - DNA, 22 bp, synthetic construct
  misc_feature 1..22: note = synthetic
  source 1..22: mol_type = other DNA
tgaggcagtaaggaacattggc

SEQ ID NO: 15 - DNA, 17 bp, synthetic construct
  misc_feature 1..17: note = synthetic
  source 1..17: mol_type = other DNA
agtccatgcctgcgcac

SEQ ID NO: 16 - DNA, 21 bp, synthetic construct
  misc_feature 1..21: note = synthetic
  source 1..21: mol_type = other DNA
gcagctctgagaaactggagg

SEQ ID NO: 17 - DNA, 21 bp, synthetic construct
  misc_feature 1..21: note = synthetic
  source 1..21: mol_type = other DNA
tccggcctcgaaagagattct

SEQ ID NO: 18 - DNA, 23 bp, synthetic construct
  misc_feature 1..23: note = synthetic
  source 1..23: mol_type = other DNA
ggcgtgaaccacgagaagtataa

SEQ ID NO: 19 - DNA, 18 bp, synthetic construct
  misc_feature 1..18: note = synthetic
  source 1..18: mol_type = other DNA
ggcagtgatggcgtggac

SEQ ID NO: 20 - DNA, 20 bp, synthetic construct
  misc_feature 1..20: note = synthetic
  source 1..20: mol_type = other DNA
cttggatcgccatacccctc

SEQ ID NO: 21 - DNA, 20 bp, synthetic construct
  misc_feature 1..20: note = synthetic
  source 1..20: mol_type = other DNA
gggctccgggttgtagattc

SEQ ID NO: 22 - DNA, 20 bp, synthetic construct
  misc_feature 1..20: note = synthetic
  source 1..20: mol_type = other DNA
gaggaccacattgtgagggt

SEQ ID NO: 23 - DNA, 21 bp, synthetic construct
  misc_feature 1..21: note = synthetic
  source 1..21: mol_type = other DNA
cgggtgatgttgtaatcccag

SEQ ID NO: 24 - DNA, 19 bp, synthetic construct
  misc_feature 1..19: note = synthetic
  source 1..19: mol_type = other DNA
cgacgaggggttgtgtctg

SEQ ID NO: 25 - DNA, 20 bp, synthetic construct
  misc_feature 1..20: note = synthetic
  source 1..20: mol_type = other DNA
acatagctattggggcagcg

SEQ ID NO: 26 - DNA, 18 bp, synthetic construct
  misc_feature 1..18: note = synthetic
  source 1..18: mol_type = other DNA
tcatccaagatggccgcc

SEQ ID NO: 27 - DNA, 20 bp, synthetic construct
  misc_feature 1..20: note = synthetic
  source 1..20: mol_type = other DNA
gccagcacttctgtttcagc

SEQ ID NO: 28 - DNA, 20 bp, synthetic construct
  misc_feature 1..20: note = synthetic
  source 1..20: mol_type = other DNA
gaaatcacggaggagtggca

SEQ ID NO: 29 - DNA, 21 bp, synthetic construct
  misc_feature 1..21: note = synthetic
  source 1..21: mol_type = other DNA
aacagctgtgaaaccacctca

SEQ ID NO: 30 - DNA, 22 bp, synthetic construct
  misc_feature 1..22: note = synthetic
  source 1..22: mol_type = other DNA
cagggaatcaatcaggtgacga

SEQ ID NO: 31 - DNA, 20 bp, synthetic construct
  misc_feature 1..20: note = synthetic
  source 1..20: mol_type = other DNA
atgggggtggaattcatcgg

SEQ ID NO: 32 - DNA, 20 bp, synthetic construct
  misc_feature 1..20: note = synthetic
  source 1..20: mol_type = other DNA
cagtgaagtgctcgtctggt

SEQ ID NO: 33 - DNA, 21 bp, synthetic construct
  misc_feature 1..21: note = synthetic
  source 1..21: mol_type = other DNA
cgactcaatcccatacaccgt

SEQ ID NO: 34 - DNA, 20 bp, synthetic construct
  misc_feature 1..20: note = synthetic
  source 1..20: mol_type = other DNA
aagccaagcttccgctatca

SEQ ID NO: 35 - DNA, 20 bp, synthetic construct
  misc_feature 1..20: note = synthetic
  source 1..20: mol_type = other DNA
cgatgacagtcaggtccgtg

(19) United States
(12) Patent Application Publication
(10) Pub. No.: US 2024/0133894 A1
(43) Pub. Date: Apr. 25, 2024

(54) BIOMARKERS FOR EARLY DIAGNOSIS AND DIFFERENTIATION OF MYCOBACTERIAL INFECTION

(71) Applicant: Wisconsin Alumni Research Foundation, Madison, WI (US)

(72) Inventors: Adel Mohamed Talaat, Madison, WI (US); Chia-wei Wu, Madison, WI (US)

(21) Appl. No.: 18/540,517

(22) Filed: Dec. 14, 2023

Related U.S. Application Data
(63) Continuation of application No. 16/555,819, filed on Aug. 29, 2019, now Pat. No. 11,860,168.
(60) Provisional application No. 62/728,387, filed on Sep. 7, 2018.

(51) Int. Cl. G01N 33/68; C12Q 1/6851; G01N 33/569

(52) U.S. Cl. CPC G01N 33/6854; C12Q 1/6851; G01N 33/5695; G01N 33/68; G01N 2800/26; G01N 2800/50

(57) ABSTRACT

Mycobacterial-specific biomarkers and methods of using such biomarkers for diagnosis of mycobacterial infection in a mammal are disclosed.

### CROSS-REFERENCE TO RELATED APPLICATIONS

This application is a continuation of U.S. Ser. No. 16/555,819, filed Aug. 29, 2019, which application claims priority to U.S. Provisional Patent Application No. 62/728,387 filed Sep. 7, 2018, which applications are incorporated herein in entireties.

### STATEMENT REGARDING FEDERALLY SPONSORED RESEARCH

This invention was made with government support under 2013-67015-21347, 2018-67015-28243 and 2016-33610-25438 awarded by the USDA/NIFA. The government has certain rights in the invention.

### REFERENCE TO A SEQUENCE LISTING SUBMITTED VIA EFS-WEB

The content of the ASCII xml file of the sequence listing named “960296.04483.xml” which is 68,144 bytes in size was created on Dec. 13, 2023 and electronically submitted via Patent Center herewith the application is incorporated herein by reference in its entirety.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BACKGROUND OF THE INVENTION

The field of the invention is vaccine and diagnostic biomarkers. More particularly, the invention relates to a set of biomarkers for diagnosing mycobacterial infection and distinguishing between vaccinated and infected animals.

Mycobacterial infections cause significant health problems to humans and animals including human tuberculosis, bovine tuberculosis, and Johne's disease. Johne's disease (aka paratuberculosis) is caused by infection with Mycobacterium avium subspecies paratuberculosis (M. ap); this disease causes severe economic losses estimated at $500 million per year for the US dairy industry alone, and these infections constitute a problem for 91% of dairy herds. Bovine tuberculosis, which is caused by infection with M. bovis, is endemic in dairy herds in several parts of the developing world and a significant problem for the wildlife animals in several developed countries (e.g., UK, USA, and Australia).

Current diagnostics can detect mycobacterial infections in cattle that have started to shed the bacteria or developed an antibody response. The available diagnostic tools are unreliable to detect early stages of infection or to differentiate infected from vaccinated animals (aka the DIVA principle). Early detection of mycobacterial infections is imperative to control the infection in herds. Further, the availability of a DIVA-based assay will facilitate adoption of new vaccines that can prevent M. ap infection.

Needed in the art are methods or diagnostic tools for detecting early stages of mycobacterial infection. Additionally, needed in the art are methods or diagnostic tools for distinguishing vaccinated from infected animals.

### SUMMARY OF THE INVENTION

The present invention overcomes the aforementioned drawbacks by providing a set of biomarkers to diagnose infected animals and distinguishing between vaccinated and infected animals.

In a first aspect, provided herein is a method for diagnosis of mycobacterial infection in a mammal, the method comprising the steps of a) obtaining a first type of sample from the mammal; b) measuring the expression level in the sample of at least one biomarker selected from the group consisting of LOC108634521, LOC108637251, LOC108637252, LOC108634594, FAM198B, LOC108637671, CDCP1, TMTC1, BAIAP2L1, MEI1, SEPT10, IFNG, IL17F, FCER2, ADGRG1, APBB1, PIWIL2, AOAH, and homologs thereof and comparing the level of the biomarker against the level detected in a first type of sample from an uninfected mammal of the same species; and c) determining the infection status of the mammal, wherein differential expression of the biomarker is indicative of a mycobacterial infection in the mammal. In some embodiments, the method is used for early diagnosis and detection of mycobacterial infection in a mammal. In some embodiments, the measuring is via ELISA assay for antibodies formed against the biomarker. In some embodiments, measuring is via quantitative PCR. In some embodiments, the sample is a blood sample. In some embodiments, the mammal is selected from the group consisting of cow, sheep and goat.

In a second aspect, provided herein is a method for differentiating between mammals who have been vaccinated with a live-attenuated mycobacterial vaccine and non-vaccinated mammals, the method comprising the steps of a) obtaining a first type of sample from the mammal; b) measuring the expression level in the sample of at least one biomarker selected from the group consisting of LOC108634521, NOS2, LOC108637251, TINAGL1, RETN, C1QL2, TDRD10, TGFB3, ADGRE2, LIPG, KCNJ2, AQP9, BPI, IL9, IL1R2, IL36B, IGF1, BGN, PIWIL2, RAET1E, CRABP2, AOAH, and homologs thereof and comparing the level of the biomarker against the level detected in a first type of sample from a non-vaccinated mammal of the same species; and c) determining the vaccination status of the mammal, wherein differential expression of the biomarker is indicative of a mammal who has been vaccinated with a live-attenuated mycobacterial vaccine. In some embodiments, the measuring is via ELISA assay for antibodies formed against the biomarker. In some embodiments, the measuring is via quantitative PCR. In some embodiment, the sample is a blood sample. In some embodiments, the mammal is selected from the group consisting of cow, sheep and goat.

In some embodiments, the live-attenuated mycobacterial vaccine is a Mycobacterium mutant vaccine. In some embodiments, the Mycobacterium mutant vaccine comprises at least one mutation in at least one gene sequence encoding global gene regulators (GGRs) selected from the group consisting of sigH, sigL and LipN.

In a third aspect, provided herein is a method for differentiating between mammals who have been vaccinated with a live-attenuated mycobacterial vaccine and infected mammals, the method comprising the steps of a) obtaining a first type of sample from the mammal; b) measuring the expression level in the sample of at least one biomarker selected from the group consisting of LOC106503226, PMP22, ART5, LOC102169116, GNLY, ASAP3, LOC108633178, TBKBP1, SLC17A7, LOC108638192, IFNG and homologs thereof and comparing the level of the biomarker against the level detected in a first type of sample from an infected mammal of the same species; and c) determining the vaccination status of the mammal, wherein differential expression of the biomarker is indicative of a mammal who has been vaccinated with a live-attenuated mycobacterial vaccine. In some embodiments, the measuring is via ELISA assay for antibodies formed against the biomarker. In some embodiments, the measuring is via quantitative PCR. In some embodiments, the sample is a blood sample. In some embodiments, the mammal is selected from the group consisting of cow, sheep and goat.

In some embodiments, the live-attenuated mycobacterial vaccine is a Mycobacterium mutant vaccine. In some embodiments, the Mycobacterium mutant vaccine comprises at least one mutation in at least one gene sequence encoding global gene regulators (GGRs) selected from the group consisting of sigH, sigL and LipN.

In a forth aspect, provided herein is a method for differentiating between mammals who have been vaccinated with a live-attenuated mycobacterial vaccine, non-vaccinated naïve mammals, and infected mammals, the method comprising the steps of obtaining a first type of sample from the mammal; measuring the expression level of FAM198B and AOAH in the sample; determining the vaccination or infection status of the mammal, wherein when relative expression of FAM198B is higher than AOAH the subject is infected, when relative expression of AOAH is higher than FAM198B the subject is vaccinated, and when the relative expression of AOAH and FAM198B are equal the subject is naïve.

In some embodiments, the measuring is via ELISA assay for antibodies formed against the biomarker. In some embodiments, the measuring is via quantitative PCR. In some embodiments, the sample is a blood sample. In some embodiments, the mammal is selected from the group consisting of cow, sheep, and goat. In some embodiments, the live-attenuated mycobacterial vaccine is a Mycobacterium mutant vaccine. In some embodiments, the Mycobacterium mutant vaccine comprises at least one mutation in at least one gene sequence encoding global gene regulators (GGRs) selected from the group consisting of sigH, sigL and LipN.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, reference is made therefore to the claims and herein for interpreting the scope of the invention.

### BRIEF DESCRIPTION OF THE DRAWINGS

The patent or application file contains at least one drawing in color. Copies of this patent or patent application publication with color drawings will be provided by the Office upon request and payment of the necessary fee.

FIG. 1 is a graph showing the alignment plot of amino acids deduced from the protein sequence in LipN of both M. paratuberculosis (SEQ ID NO:1) and M. bovis (SEQ ID NO:2). The consensus sequence is SEQ ID NO:3. Peptides conserved in M. paratuberculosis sequence but absent from M. bovis were targets for previous DIVA testing methods.

FIGS. 2A-2B is a diagram showing multiplex PCR strategy using 3 primers. (A) Wild-type (virulent) strain with intact lipN gene. (B) LAV strain with scar sequence from hygromycin cassette removal represented by the black rectangle.

FIGS. 3A-3C show RNA sequencing analysis of different goat groups following infection or vaccination with LAV or Mycopar vaccines. MAplots of (A) the infected group compared to the naïve group, (B) the LAV-vaccinated group compared to the naïve group and (C) the Mycopar-vaccinated group compared to the naïve group are shown. Red dots represent differentially expressed transcripts (fold change >2.0 or <−2.0, p<0.05).

FIGS. 4A-4C show comparative transcriptome analysis of the infected and vaccinated goat groups. Venn diagrams show numbers of common DE transcripts between (A) the infected and vaccinated groups, each compared to the naïve group. (B) infected vs. naïve and infected vs. LAV-vaccinated groups and (C) LAV-vaccinated vs. naïve and Mycopar-vaccinated vs. naïve groups.

FIG. 5 shows significant terms in gene ontology analysis for the differentially expressed genes in infected goats compared to naïve control goats. The agriGO for automated identification of GO terms were used on the list of genes with significant differential expression when the transcriptomes of infected and naïve goats were compared.

FIGS. 6A-6B show gene network analysis of DE genes in the infected group. (A) Genes that were significantly up-regulated. (B) Genes that were significantly down-regulated. IL-17A and IL-17F are two homologs in the IL-17 family. Light green lines represent connection between genes co-mentioned in an abstract in published studies, cyan lines represent putative pathway connections found in homologs in other species, black lines represent co-expression in Bos taurus or homologs in other species and pink lines represent experimentally determined association.

FIGS. 7A-7D show the transcriptional profile of key caprine genes over 12 months post challenge with M. paratuberculosis. Panels display quantitative real-time PCR analysis of total RNA extracted from blood samples collected from goat groups at 1 (A), 2 (B), 6 (C) and 12 (D) months post challenge (MPC). Expression levels were calculated with ΔΔCt relative quantitation method relative to the GAPDH gene expression in the naïve group. Target gene names are listed below each panel and fold change for the infected, Mycopar® or LAV-vaccinated relative to naïve goat groups are listed on the Y-axis. At each time point, samples from three animals in each group except the infected group at 1 MPC (N=2) were included and standard errors of the mean (SEM) of the three measurements were presented as error bars.

FIGS. 8A-8C show significant terms in gene ontology analysis, using agriGO, for the differentially expressed genes. The significant terms for the GO analysis for the Mycopar-vaccinated vs naïve group is shown in (A). Chart in (B) shows the significant terms for LAV-vaccinated vs naïve group and in (C) shows the significant terms for LAV-vaccinated vs. infected group. The significant GO terms for both comparisons are 1-binding, 2-intracellular, 3-intracellular part and 4-metabolic process.

### DETAILED DESCRIPTION OF THE INVENTION

The term “Mycobacterium,” as used herein, refers to a genus of actinobacteria given its own family, the mycobacteriaceae. The genus includes pathogens known to cause serious diseases in mammals, including tuberculosis (Mycobacterium tuberculosis) and leprosy (Mycobacterium leprae).

Mycobacterium tuberculosis complex (MTBC) members are causative agents of human and animal tuberculosis. Species in this complex may include M. tuberculosis, the major cause of human tuberculosis, M. bovis, M. bovis BCG, M. africanum, M. canetti, M. caprae, M. microti, and M. pinnipedii.

Mycobacterium avium complex (MAC) is a group of species that, in a disseminated infection but not lung infection, used to be a significant cause of death in AIDS patients. Species in this complex include M. avium, M. avium paratuberculosis, which has been implicated in Crohn's disease in humans and is the causative agent of Johne's disease in cattle and sheep, M. avium silvaticum, M. avium “hominissuis,” M. colombiense, and M. indicus pranii.

Mycobacterial infections are notoriously difficult to treat. The organisms are hardy due to their cell wall, which is neither truly Gram negative nor Gram positive. In addition, they are naturally resistant to a number of antibiotics that disrupt cell-wall biosynthesis, such as penicillin. Due to their unique cell wall, they can survive long exposure to acids, alkalis, detergents, oxidative bursts, lysis by complement, and many antibiotics. Most mycobacteria are susceptible to the antibiotics clarithromycin and rifamycin, but antibiotic-resistant strains have emerged.

The term “biomolecule,” as used herein, refers to any organic molecule that is part of or from a living organism. Biomolecules may include nucleic acids, a nucleotide, a polynucleotide, an oligonucleotide, a peptide, a protein, a carbohydrate, a ligand, a receptor, among others. In one embodiment of the present invention, biomolecules may include genes and their expression products.

The term “expression product,” as used herein, refers to any product produced during the process of gene expression. These products are often proteins, but in non-protein coding genes such as ribosomal RNA (rRNA), transfer RNA (tRNA) or small nuclear RNA (snRNA) genes, the product is a functional RNA.

The terms “polypeptide,” “peptide,” and “protein,” as used herein, refer to a polymer comprising amino acid residues predominantly bound together by covalent amide bonds. The terms apply to amino acid polymers in which one or more amino acid residue may be an artificial chemical mimetic of a naturally occurring amino acid, as well as to naturally occurring amino acid polymers and non-naturally occurring amino acid polymers. As used herein, the terms may encompass amino acid chains of any length, including full length proteins, wherein the amino acids are linked by covalent peptide bonds. The protein or peptide may be isolated from a native organism, produced by recombinant techniques, or produced by synthetic production techniques known to one skilled in the art.

The term “recombinant protein,” as used herein, refers to a polypeptide of the present disclosure which is produced by recombinant DNA techniques, wherein generally, DNA encoding a polypeptide is inserted into a suitable expression vector which is in turn used to transform a heterologous host cell (e.g., a microorganism or yeast cell) to produce the heterologous protein.

The term “recombinant nucleic acid” or “recombinant DNA,” as used herein, refers to a nucleic acid or DNA of the present disclosure which is produced by recombinant DNA techniques, wherein generally, DNA encoding a polypeptide is inserted into a suitable expression vector which is in turn used to transform a host cell to produce the heterologous protein.

The term “mammal,” as used herein, refers to any living species which can be identified by the presence of sweat glands, including those that are specialized to produce milk to nourish their young. In one embodiment, the mammal suitable for the present invention may include bubaline, elephantine, musteline, pardine, phocine, rhinocerine, caprine, hircine, leonine, leporine, lupine, lyncine, murine, rusine, tigrine, ursine, vulpine, zebrine, vespertilionine, porcine, bovine, equine, swine, elaphine, ovine, caprine, camelidae, feline, cervine, primate, human and canine mammals. In one preferred embodiment of the present invention, the mammal may be one of the ruminants such as cattle, goats, sheep, giraffes, yaks, deer, camels, llamas, antelope, and some macropods. In one specific embodiment of the present invention, the mammal may include any of the milk cattle species, such as cow, sheep and goat.

The term “antibody,” as used herein, refers to a class of proteins that are generally known as immunoglobulins. The term “antibody” herein is used in the broadest sense and specifically includes full-length monoclonal antibodies, polyclonal antibodies, multispecific antibodies (e.g., bispecific antibodies), and antibody fragments, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

The term “marker” or “biomarker,” as used herein, refers to a biomolecule (e.g., protein, nucleic acid, carbohydrate, or lipid) that is differentially expressed in the cell, differentially expressed on the surface of an infected cell, differentially phosphorylated, or differentially secreted by a infected cell in comparison to a normal cell or in a paracrine fashion by neighboring uninfected cells, and which is useful for the diagnosis of mycobacterial infection, differentiating between infected and vaccinated animals, and for preferential targeting of a pharmacological agent to an infected mammal. In some embodiments, the biomarker is differentially expressed in an infected subject in comparison to a normal subject. In some embodiments, the biomarker is differentially expressed in a vaccinated subject in comparison to a non-vaccinated subject. In some embodiments, the biomarker is differentially expressed in a vaccinated subject in comparison to an infected subject.

The term “differentially expressed,” as used herein, refers to a change in expression of at least 2-fold. In some embodiments, differential expression indicates that a given biomarker is over-expressed in a first subject or cell in comparison to a second subject or cell, for instance, at least 2-fold over-expression, at least 3-fold over-expression, at least 4-fold over-expression or more in comparison to the second subject or cell. In some embodiments, differential expression indicates that a given biomarker has decreased expression in a first subject or cell in comparison to a second subject or cell, for instance, at least a 2-fold decease in expression, at least a 3-fold decrease in expression, at least a 4-fold decrease in expression or more.

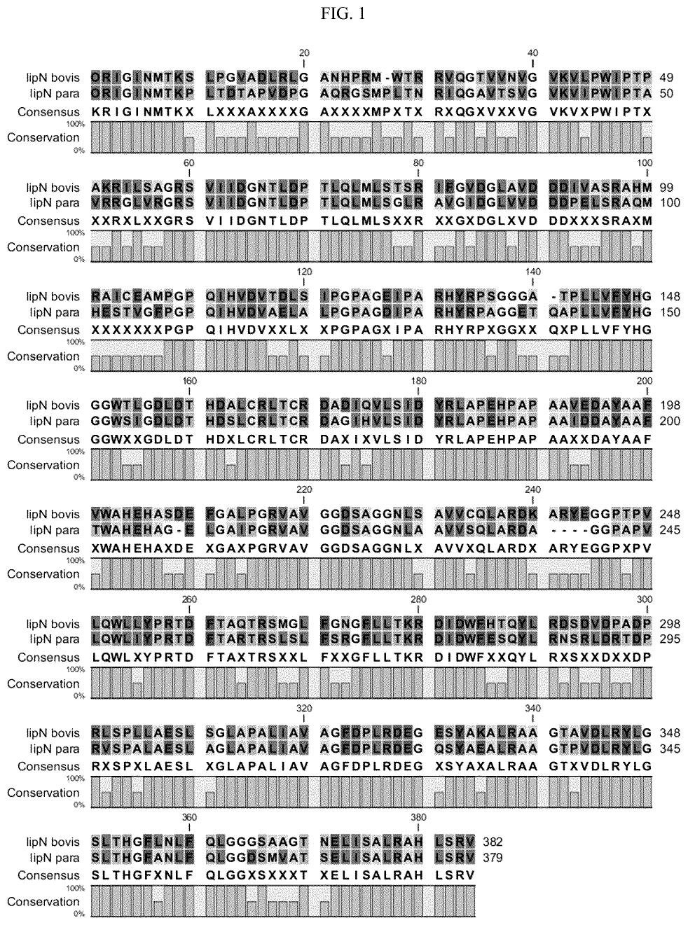

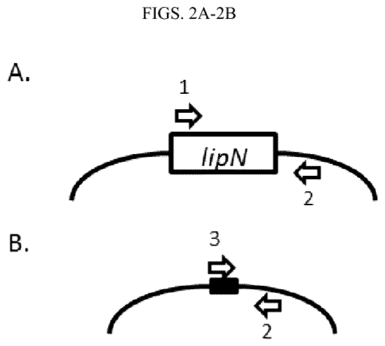

The term “mycobacterial-specific biomarkers,” as used herein, refers to biomarkers which are specifically related to mycobacterial infection. Some of these biomarkers are listed in FIG. 1 and Tables 4 and 5.

The term “lyophilization,” as used herein, refers to freezing of a material at low temperature followed by dehydration by sublimation, usually under a high vacuum. Lyophilization is also known as freeze drying. Many techniques of freezing are known in the art of lyophilization such as tray freezing, shelf freezing, spray-freezing, shell-freezing and liquid nitrogen immersion. Each technique will result in a different rate of freezing. Shell freezing may be automated or manual. For example, flasks can be automatically rotated by motor driven rollers in a refrigerated bath containing alcohol, acetone, liquid nitrogen, or any other appropriate fluid. A thin coating of product is evenly frozen around the inside “shell” of a flask, permitting a greater volume of material to be safely processed during each freeze drying run. Tray freezing may be performed by, for example, placing the samples in lyophilizer, equilibrating 1 hr at a shelf temperature of 0° C., then cooling the shelves at 0.5° C./min to −40° C. Spray-freezing, for example, may be performed by spray freezing into liquid, dropping by ˜20 μl droplets into liquid N₂, spray freezing into vapor over liquid, or by other techniques known in the art.

The term “antigen,” as used herein, refers to any molecule that is capable of eliciting an immune response, whether a cell-mediated or humoral immune response, whether in the presence or absence of an adjuvant. An antigen can be any type of molecule, e.g., a peptide or protein, a nucleic acid, a carbohydrate, a lipid, and combinations thereof. A “vaccine antigen” is an antigen that can be used in a vaccine preparation. A “therapeutic antigen” is an antigen that can be used for therapeutic purposes.

The term “vaccine,” as used herein, refers to an antigenic preparation used to produce active immunity to a disease, in order to prevent or ameliorate the effects of infection. The antigenic moiety making up the vaccine may be either a live or killed microorganism, or a natural product purified from a microorganism or other cell including, but not limited to tumor cells, a synthetic product, a genetically engineered protein, peptide, polysaccharide or similar product or an allergen.

The term “immunologically active,” as used herein, refers to the ability to raise one or more of a humoral response or a cell mediated response specific to an antigen.

The term “adjuvant,” as used herein, refer to compounds that, when used in combination with specific vaccine antigens in formulations, augment or otherwise alter or modify the resultant immune responses. An adjuvant combined with a vaccine antigen increases the immune response to the vaccine antigen over that induced by the vaccine antigen alone. An adjuvant may augment humoral immune responses or cell-mediated immune responses or both humoral and cell-mediated immune responses against vaccine antigens.

The term “detecting,” as used herein, refers to confirming the presence of the biomarker or marker present in the sample. Quantifying the amount of the biomarker or marker present in a sample may include determining the concentration of the biomarker present in the sample. Detecting and/or quantifying may be performed directly on the sample, or indirectly on an extract therefrom, or on a dilution thereof.

The term “homology,” as used herein, refers to the resemblance or similarity between two nucleotide or amino acid sequences. As applied to a gene, “homolog” may refer to a gene similar in structure and/or evolutionary origin to a gene in another organism or another species. As applied to nucleic acid molecules, the term “homolog” means that two nucleic acid sequences, when optimally aligned (see below), share at least 80 percent sequence homology, preferably at least 90 percent sequence homology, more preferably at least 95, 96, 97, 98 or 99 percent sequence homology. “Percentage nucleotide (or nucleic acid) homology” or “percentage nucleotide (or nucleic acid) sequence homology” refers to a comparison of the nucleotides of two nucleic acid molecules which, when optimally aligned, have approximately the designated percentage of the same nucleotides or nucleotides that are not identical but differ by redundant nucleotide substitutions (the nucleotide substitution does not change the amino acid encoded by the particular codon). For example, “95% nucleotide homology” refers to a comparison of the nucleotides of two nucleic acid molecules which, when optimally aligned, have 95% nucleotide homology.

In one aspect, the present invention relates to a method for diagnosis of mycobacterial infection in a mammal. In one embodiment, the present invention discloses a method for early detection of mycobacterial infection. The term “early detection,” as used herein, refers to detection of mycobacterial infection during the early stage of infection, e.g., a stage before the development of chronic diarrhea.

In another embodiment, the present invention discloses a method for differentiating a vaccinated mammal from a non-infected mammal or a mycobacterial infected mammal.

The detection of mycobacterial infection and related diseases such as Johne's disease is very difficult because the disease generally takes many years to develop, and the organism is shed by the mammal periodically, so every mammal must be repeatedly tested over long time periods.

Applicants have identified mycobacterial-specific biomarkers and host-specific biomarkers, such as genes and/or expression products derived thereof, useful for detection of mycobacterial infection. Mycobacterial-specific biomarkers, host-specific biomarkers or a combination of such biomarkers may also be used to differentiate a vaccinated mammal (e.g., from genetically engineered vaccines) from a non-infected mammal or a mycobacterial-infected mammal.

### Differentiating Vaccinated Mammals from Mycobacterial-Infected Mammals

In one embodiment, the present invention discloses a method for differentiating a vaccinated mammal from a mycobacterial infected mammal.

In one embodiment, the method for differentiating a vaccinated mammal from a mycobacterial-infected mammal comprises the steps of (a) obtaining a sample from the mammal; (b) testing the sample for the concentration level of at least one biomarker and comparing the level of the biomarker against the level detected in an infected mammalian sample; and (c) determining the infection or vaccination status of the mammal.

A sample suitable for the present invention may include any biological sample from the mammal. The biological sample may include, without limitation, saliva, sputum, blood, plasma, serum, urine, feces, cerebrospinal fluid, amniotic fluid, wound exudate, or tissue of the subject of mammal. In one specific embodiment, the biological sample is a blood sample. In some embodiments, when comparing the expression level of a biomarker between two subjects, it is desirable to compare expression levels in the same type of sample.

A major problem in employing mass vaccination program for the control of Johne's disease in dairy herds is the inability to differentiate between infected and vaccinated animals with the current vaccine (DIVA principal). Applicants have previously proposed using genetically engineered vaccines (PCT patent application publication WO2014164055, U.S. Pat. Nos. 9,663,758, and 9,446,110, each of which is incorporated herein by reference). One would wish to consider the DIVA principal and wish to distinguish between M. paratuberculosis infected and Johne's disease vaccinated animals that have been vaccinated with genetically engineered vaccines.

In one embodiment, Applicants identify biomolecules as mycobacterial-specific biomarkers. For example, the biomolecules of mycobacterial-specific biomarkers may include genes and their expression products which are present in a M. ap wild-type strain but not present or have a low expression level in genetically engineered vaccines and vaccinated animals. In one embodiment involving sigL and sigH mutants, the mycobacterial-specific biomarker may comprise at least one member selected from the group consisting of gene sequences Q73SF4, Q73Y73, Q73ZE6, Q73SL7, Q73VK6, Q73XZ0, Q740D1 and Q73UE0 and expression products derived thereof. In another embodiment, the mycobacterial-specific biomarker may comprise at least two, three, four, five, six, seven or eight members selected from the group consisting of gene sequences Q73SF4, Q73Y73, Q73ZE6, Q73SL7, Q73VK6, Q73XZ0, Q740D1 and Q73UE0 and expression products derived thereof. Preferably, the mycobacterial-specific biomarker may comprise at least two members selected from the group as discussed above.

In one embodiment involving sigL and sigH mutants, the mycobacterial-specific biomarker comprises at least one member selected from the group consisting of gene sequences Q73VL6, Q73YW9, Q741L4, Q744E5, Q73YP5, Q73WE5, Q73U21, Q73UH9, Q741M5, Q742F4, and Q73SU6 and expression products derived thereof. In one embodiment, the mycobacterial-specific biomarker comprises at least two, three, four, five, six, seven, eight, nine or ten members selected from the group consisting of gene sequences Q73VL6, Q73YW9, Q741L4, Q744E5, Q73YP5, Q73WE5, Q73U21, Q73UH9, Q741M5, Q742F4, and Q73SU6 and expression products derived thereof.

Applicants envision that the present invention may be applicable to any genetically engineered vaccines. In some embodiments, the vaccine may be an inactived vaccine (e.g., Mycopar™). In one specific embodiment, the present invention is applicable to live attenuated vaccines. The example of the live attenuated vaccines may include sigL, sigH, or LipN mutants. PCT patent application publication WO20141640055A1 discloses live attenuated vaccines, such as sigL and sigH mutants. PCT patent application publication WO20141640055A1 is incorporated herein by reference in its entirety.

In one embodiment, the presence or absence of the biomarkers in a mammal may demonstrate the infection status of the mammal. In one specific embodiment, the biomarkers that are significantly over-expressed in the wild type strain and not in the mutant vaccine and could be used for the mutant vaccine-DIVA testing.

For example, when the biomarkers are those significantly over-expressed in the wild type strain and not in the mutant vaccine, the presence of at least one biomarker in a mammal shows that the mammal may be infected and not merely vaccinated. On the other hand, the absence of at least one biomarker in a mammal shows that the mammal may be vaccinated.

In one embodiment, Applicants envision that the present invention is also applicable when antigens are inoculated to a mammal and the infection status of the mammal needs to be identified. Specifically, the infection status may include whether a mammal is vaccinated or whether a mammal is infected with M. paratuberculosis.

Table 7, which tabulates the result of one of the Examples drawn to host transcriptome analysis of goats, lists additional markers that will be useful for embodiments of the invention. The first part of Table 7 lists DNA markers that are useful for early diagnosis of John's disease in ruminants, as described above, because the markers differentiate infected from naïve animals. Table 7 lists the locus in goats and provides homologous locus in cows, if it is known. Table 7 also lists host markers that can differentiate live attenuated vaccine (LAV) vaccinated animals from naïve animals and markers that can differentiate inactivated-vaccine immunized from naïve animals. Table 7 also lists host markers to distinguish between infected and vaccinated animals.

As used herein, “naïve” refers to animals that are not vaccinated nor infected.

In addition to the biomarkers recited herein, additional biomarkers useful in the disclosed methods include those described in U.S. Pat. No. 10,054,586, which is incorporated herein in its entirety.

In some embodiments, the present invention provides methods of distinguishing between infected and naïve animals by measuring expression of one or more biomarkers selected from the group of LOC108634521, LOC108637251, LOC108637252, LOC108634594, FAM198B, LOC108637671, CDCP1, TMTC1, BAIAP2L1, MEI1, SEPT10, IFNG, IL17F, FCER2, ADGRG1, APBB1, PIWIL2, AOAH, and homologs thereof. (Table 1) In some embodiments, two, three, four, five, six or more of the recited biomarkers may be measured.

In some embodiments, the present invention provides methods of distinguishing between live attenuated vaccine (LAV) vaccinated animals from naïve animals by measuring expression of one or more biomarker selected from the group of LOC108634521, NOS2, LOC108637251, TINAGL1, RETN, C1QL2, TDRD10, TGFB3, ADGRE2, LIPG, KCNJ2, AQP9, BPI, IL9, IL1R2, IL36B, IGF1, BGN, PIWIL2, RAET1E, CRABP2, AOAH, and homologs thereof. (Table 2) In some embodiments, two, three, four, five, six or more of the recited biomarkers may be measured.

In some embodiments, the present invention provides methods of distinguishing between LAV-vaccinated animals from infected animals by measuring expression of one or more biomarker selected from the group of LOC106503226, PMP22, ART5, LOC102169116, GNLY, ASAP3, LOC108633178, TBKBP1, SLC17A7 and homologs thereof. (Table 3) In some embodiments, two, three, four, five, six or more of the recited biomarkers may be measured.

In some embodiments, biomarkers may be used to distinguish between naïve, infected, and vaccinated animals. By measuring expression of two or more biomarkers recited herein, an animal may be identified as naïve, infected, or vaccinated. A selection of suitable biomarkers and their relative expression is outlined in Table 4A and Table 4B. For example, as demonstrated in Table 4A, when relative expression of FAM198B is higher than relative expression of AOAH, the subject is infected with a mycobacterial infection. When relative expression of AOAH is higher than relative expression of FAM198B, the subject has been vaccinated with an LAV vaccine. When relative expression of AOAH and FAM198B are equal, or within error of the method used to quantify relative expression, the subject is naïve. Similar comparisons and conclusions may be drawn using other biomarkers described herein, such as those outlined in Table 4B or any of Tables 1-3.

TABLE 1
List of host (goat and cow) genes that can differentiation infected from naïve animals.

| | | | | | Log₂ |
|---|---|---|---|---|---|
| | | | Expression in | Expression in | Fold Change in |
| | Entrez | | a Naïve | an Infected | Expression |
| Symbol | Gene ID | Description | Animal | Animal | Infected vs Naïve |
| LOC108634521 | 108634521 | non-coding RNA | 0.66 | 258.7 | 8.66 |
| LOC108637251 | 108637251 | multidrug resistance-associated protein 4-like | 1.28 | 137.44 | 6.74 |
| LOC108637252 | 108637252 | multidrug resistance-associated protein 4-like | 5.31 | 90.39 | 4.11 |
| LOC108634594 | 108634594 | multidrug resistance-associated protein 4-like | 3.35 | 110.84 | 5.06 |
| FAM198B | 102191727 | family with sequence similarity 198 member B | 71.81 | 768.85 | 3.42 |
| LOC108637671 | 108637671 | tripartite motif-containing protein 5-like | 46.57 | 316.63 | 2.77 |
| CDCP1 | 102187276 | CUB domain containing protein | 8.9 | 36.66 | 2.02 |
| TMTC1 | 102185637 | transmembrane and tetratricopeptide repeat | 13.2 | 47.85 | 1.86 |
| BAIAP2L1 | 102173150 | BAI1 associated protein 2 like | 8.17 | 29.54 | 1.85 |
| MEI1 | 102169168 | meiotic double-stranded break formation protein | 203.8 | 609.18 | 1.58 |
| SEPT10 | 102171885 | septin 10 | 16.83 | 40.16 | 1.27 |
| IFNG | 100860815 | interferon gamma | 63.77 | 19.26 | −1.75 |
| IL17F | 102171111 | interleukin 17F | 625.76 | 267.69 | −1.22 |
| FCER2 | 102171507 | Fc fragment of IgE receptor II | 626.49 | 243.68 | −1.36 |
| ADGRG1 | 102171366 | G protein-coupled receptor G | 72.43 | 21.2 | −1.78 |
| APBB1 | 102179305 | amyloid beta precursor protein binding family B member | 97.39 | 21.75 | −2.16 |
| PIWIL2 | 102173845 | piwi like RNA-mediated gene silencing | 57.32 | 10.67 | −2.41 |
| AOAH | 102189546 | acyloxyacyl hydrolase | 258.05 | 268.21 | 0.06 |

TABLE 2
List of host (goat and cow) genes that can differentiate LAV vaccinated from naïve animals.

| | | | | | Log₂ |
|---|---|---|---|---|---|
| | | | Expression in | Expression in an | Fold Change in |
| | Entrez | | a Naïve | LAV vaccinated | Expression |
| Symbol | Gene ID | Description | Animal | Animal | Vaccinated vs Naïve |
| LOC108634521 | 108634521 | non-coding RNA | 0.66 | 238.29 | 8.54 |
| NOS2 | 100860742 | nitric oxide synthase 2 | 0.78 | 103.4 | 7.03 |
| LOC108637251 | 108637251 | multidrug resistance-associated protein 4-like | 1.28 | 150.28 | 6.87 |
| TINAGL1 | 102169636 | tubulointerstitial nephritis antigen like | 4.62 | 90.01 | 4.27 |
| RETN | 102170965 | resistin | 44.98 | 708.35 | 3.96 |
| C1QL2 | 102176742 | complement C1q like 2 | 2.29 | 30.2 | 3.69 |
| TDRD10 | 102174259 | tudor domain containing 10 | 0 | 2.49 | 3.53 |
| TGFB3 | 102189962 | transforming growth factor beta 3 | 40.44 | 367.3 | 3.19 |
| ADGRE2 | 102171592 | adhesion G protein-coupled receptor E2 | 101.97 | 601.2 | 2.56 |
| LIPG | 102191574 | lipase G endothelial type | 33.18 | 175.34 | 2.40 |
| KCNJ2 | 102168940 | potassium voltage-gated channel subfamily J member 2 | 78.28 | 378.07 | 2.27 |
| AQP9 | 102181396 | aquaporin 9 | 72.94 | 342.39 | 2.24 |
| BPI | 102185756 | bactericidal/permeability-increasing protein | 13.67 | 49.85 | 1.85 |
| IL9 | 102179848 | interleukin 9 | 13.72 | 4.82 | −1.54 |
| IL1R2 | 102186601 | interleukin 1 receptor type 2 | 170.86 | 47.04 | −1.86 |
| IL36B | 102182235 | interleukin 36 beta | 80.82 | 14.69 | −2.45 |
| IGF1 | 100860838 | insulin like growth factor | 109.36 | 19.9 | −2.45 |
| BGN | 102183219 | biglycan | 227.52 | 26.44 | −3.10 |
| PIWIL2 | 102173845 | piwi like RNA-mediated gene silencing | 57.32 | 7 | −3.10 |
| RAET1E | 108636743 | retinoic acid early transcript | 832.23 | 48.24 | −4.11 |
| CRABP2 | 102174348 | cellular retinoic acid binding protein 2 | 103.46 | 3.31 | −4.91 |
| AOAH | 102189546 | acyloxyacyl hydrolase | 258.05 | 1014.3 | 1.98 |

TABLE 3
List of host (goat and cow) genes that can differentiate LAV vaccinated from infected animals.

| | | | | | Log₂ |
|---|---|---|---|---|---|
| | | | Expression in | Expression in an | Fold Change in |
| | Entrez | | an Infected | LAV vaccinated | Expression |
| Symbol | Gene ID | Description | Animal | Animal | Infected vs Vaccinated |
| LOC106503226 | 106503226 | non-coding RNA | 37.38 | 5.83 | 2.74 |
| PMP22 | 102184371 | peripheral myelin protein 22 | 268.96 | 36.55 | 2.87 |
| ART5 | 102169686 | ADP-ribosyltransferase 5 | 16.98 | 103.7 | −2.62 |
| LOC102169116 | 102169116 | ecto-ADP-ribosyltransferase 5 | 43.11 | 198.79 | −2.20 |
| GNLY | 102191341 | granulysin | 32.21 | 149.78 | −2.21 |
| ASAP3 | 102182646 | ArfGAP with SH3 domain | 15.62 | 70.26 | −2.18 |
| | | ankyrin repeat and PH domain 3 | | | |
| LOC108633178 | 108633178 | granzyme B-like | 6.6 | 50.4 | −2.98 |
| TBKBP1 | 102172659 | TBK1 binding protein | 74.03 | 499.27 | −2.76 |
| SLC17A7 | 102169042 | solute carrier family 17 member 7 | 3.1 | 53.98 | −4.10 |
| FAM198B | 102191727 | family with sequence similarity 198 | 768.85 | 311.61 | 1.30 |
| | | member B | | | |
| AOAH | 102189546 | acyloxyacyl hydrolase | 268.21 | 1014.3 | −1.92 |

TABLE 4A
Use of host genes, for example, as measured by quantitative
PCR, to differentiate between naïve, infected, and
vaccinated animals

| | Relative | Relative |
|---|---|---|
| | expression | expression |
| Status of the Animal | of FAM198B | of AOAH |
| Naïve | 1 | 1 |
| Infected | 6.89 | 1.10 |
| Vaccinated with LAV vaccine | 3.49 | 10.47 |

TABLE 4B
Summary of biomarkers for use in differentiation
of infected and vaccinated animals.

| | FAM198B | AOAH | MEI1 | IL-22 | CDCP1 |
|---|---|---|---|---|---|
| Infected with MAP vs. | 6.89 | 1.1 | −1.58 | −3.84 | −1.05 |
| Naïve animas | | | | | |
| Vaccinated with | 3.49 | 10.47 | −3.17 | −1.29 | 1.64 |
| LAV vaccine vs. | | | | | |
| Naïve Animals | | | | | |

Applicants envision that the biomarker may include genes or the polynucleotides containing less than an entire gene sequence of the above genes. The biomarker of genes or the polynucleotides may be either single-or double-stranded nucleic acids. A polynucleotide may be RNA, DNA, cDNA, genomic DNA, chemically synthesized RNA or DNA or combinations thereof. The polynucleotides can be purified free of other components, such as proteins, lipids and other polynucleotides. For example, the polynucleotide may be 50%, 75%, 90%, 95%, 96%, 97%, 98%, 99%, or 100% purified. The purified polynucleotides may comprise additional heterologous nucleotides. The purified polynucleotides of the invention can also comprise other nucleotide sequences, such as sequences coding for linkers, primer, signal sequences, TMR stop transfer sequences, transmembrane domains, or ligands.

The gene or the polynucleotides of the invention may also comprise fragments that encode immunogenic polypeptides. Polynucleotides of the invention may encode full-length polypeptides, polypeptide fragments, and variant or fusion polypeptides. Polynucleotides of the invention may comprise coding sequences for naturally occurring polypeptides or may encode altered sequences that do not occur in nature. If desired, polynucleotides may be cloned into an expression vector comprising expression control elements, including for example, origins of replication, promoters, enhancers, or other regulatory elements that drive expression of the polynucleotides of the invention in host cells.

### Detection of Biomarkers or Markers

The present biomarkers or markers may be detected by any suitable method. In one embodiment, the testing is via ELISA assay for antibodies formed against the biomarkers or markers.

The biomarker or marker in the present invention may be directly detected, e.g., by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

For example, detecting and/or quantifying may be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques may include ICAT® (Applied Biosystems, CA, USA), or iTRAQ® (Applied Biosystems, CA, USA). Liquid chromatography (e.g., high pressure liquid chromatography (HPLC) or low pressure liquid chromatography (LPLC)), thin-layer chromatography, NMR (nuclear magnetic resonance) spectroscopy may also be used. Methods of diagnosing and/or monitoring according to the invention may comprise analyzing a plasma, serum or whole blood sample by a sandwich immunoassay to detect the presence or level of the biomarker. These methods are also suitable for clinical screening, prognosis, monitoring the results of therapy, identifying patients most likely to respond to a particular therapeutic treatment, for drug screening and development, and identification of new targets for drug treatment.

Detecting and/or quantifying the biomarkers or markers may be performed using an immunological method, involving an antibody, or a fragment thereof capable of specific binding to the biomarker. Suitable immunological methods include sandwich immunoassays, such as sandwich ELISA, in which the detection of the analyte biomarkers is performed using two antibodies which recognize different epitopes on a analyte biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g., using gold, silver, or latex particles, magnetic particles, or Q-dots). Immunological methods may be performed, for example, in microtiter plate or strip format.

The gene or the polynucleotides of the invention may be detected by, for example, a probe or primer or a PCR primer. The gene or the polynucleotides of the invention may be the basis for designing a complimentary probe or primer, to detect the presence and/or quantity of biomarker in a subject, such as a biological sample. Probes are molecules capable of interacting with a target nucleic acid, typically in a sequence specific manner, for example, through hybridization. Primers are a subset of probes that can support specific enzymatic manipulation and that can hybridize with a target nucleic acid such that the enzymatic manipulation occurs. A primer may be made from any combination of nucleotides or nucleotide derivatives or analogs available in the art that do not interfere with the enzymatic manipulation. “Specific” means that a gene sequence recognizes or matches another gene of the invention with greater affinity than to other non-specific molecules. Preferably, “specifically binds” or “specific to” also means a gene sequence recognizes and matches a gene sequence comprised in a biomarker described herein, with greater affinity than to other non-specific molecules.

The hybridization of nucleic acids is well understood in the art. Typically a primer may be made from any combination of nucleotides or nucleotide derivatives or analogs available in the art. The ability of such primers to specifically hybridize to polynucleotide sequences of the recited biomarkers will enable them to be of use in detecting the presence of complementary sequences in a given subject. The primers of the invention may hybridize to complementary sequences in a subject such as a biological sample, including, without limitation, saliva, sputum, blood, plasma, serum, urine, feces, cerebrospinal fluid, amniotic fluid, wound exudate, or tissue of the subject. Polynucleotides from the sample can be, for example, subjected to gel electrophoresis or other size separation techniques or can be immobilized without size separation.

The probes or the primers may also be labeled for the detection. Suitable labels, and methods for labeling primers are known in the art. For example, the label may include, without limitation, radioactive labels, biotin labels, fluorescent labels, chemiluminescent labels, bioluminescent labels, metal chelator labels and enzyme labels. The polynucleotides from the sample are contacted with the probes or primers under hybridization conditions of suitable stringencies. Preferably, the primer is fluorescently labeled. Also, the detection of the presence or quality of the gene sequence of interest can be accomplished by any method known in the art. For instance, the detection can be made by a DNA amplification reaction. In some embodiments, “amplification” of DNA denotes the use of polymerase chain reaction (PCR) to increase the concentration of a particular DNA sequence within a mixtures of DNA sequences.

In some embodiments, quantitative polymerase chain reaction (qPCR) or real-time quantitative polymerase chain reaction (RT-qPCR) is used to measure expression levels of nucleotide biomarkers. These methods detect and quantify the products generated during each cycle of the PCR process which are directly proportionate to the amount of the messenger RNA, DNA, or cDNA prior to the start of the PCR process. Some qPCR and RT-qPCR methods may use non-specific fluorescent dyes that intercalate with any double stranded DNA or sequence specific DNA probes with fluorescently labeled oligonucleotides to permit detection only after hybridization of the probe with the complementary sequence. Suitable RT-qPCR and qPCR methods, probes and dyes are known in the art.

In another embodiment, the amplification of DNA may be done by the loop-mediated isothermal amplification (LAMP). Similar to PCR, LAMP utilizes a polymerization-based reaction to amplify DNA from examined samples, but the enzyme for LAMP, Bst DNA polymerase large fragment, possesses a DNA strand displacement activity. This makes the DNA extension step possible without having to fully denature DNA templates. Moreover, the primers are designed in a way that a hairpin loop structure is formed in the first cycle of amplification, and the following products are further amplified in an auto-cycling manner. Therefore, in about an hour, the repeated reactions can amplify by ˜10⁹ copies of DNA molecules and can be done at a constant temperature in a single heat block, instead of at various cycles of temperature in a relatively expensive thermal cycler. The detection of LAMP has been described in PCT patent application publication WO20141640055A1, which is incorporated herein in its entirety.

In one embodiment, the detection of the presence of the gene or the specific binding between the gene in Mycobacterium mutant and a gene that is not a component of a subject's immune response to a particular vaccine may indicate a natural or experimental Mycobacterium infection. For example, the absence of such binding or presence may indicate the absence of Mycobacterium infection. Or, a second, separate gene, such as a mutated Mycobacterium gene that is specific to a component of a mammal's immune response to a particular Mycobacterium vaccine, may be used to detect corresponding antibodies produced in response to vaccination. Thus, if an antibody specific to a gene in Mycobacterium vaccine is detected, then the mammal has been vaccinated and/or infected. The detection of neither genes indicates no infection and no vaccination. As such, various combinations can lead to a determination of the vaccination and/or infection status of the mammal.

### Kits of the Present Invention

In another aspect, the present invention discloses a diagnostic kit suitable for carrying out the diagnostic method of the first aspect of the invention. In one embodiment, the kit may be a “one-day” kit, meaning that it is capable of providing the diagnostic result within one day of sample collection. In another embodiment, the kit may be able to provide a diagnostic result within 12, 10, 8, 6, 4, 2, 1 or 0.5 hours of sample collection.

In one embodiment, the diagnostic kit may be used for early detection of mycobacterial infection in a mammal. The diagnostic kit may also be used to differentiate a vaccinated mammal from an infected mammal.

In one embodiment, the diagnostic kit may be portable. The portable diagnostic kit may specifically suitable for field testing. Applicants envision that the present diagnostic kit may be used in a farm field such as a milk farm, where farmers/veterinarians may collect samples and run the assay on the field (point of care assay) to identify early stages of Johne's disease infection and to differentiate infected from vaccinated mammals.

The kit may include a substrate. In one embodiment, the substrate may be coated with biomolecules such as antibodies, which are specifically binding to the specific biomarkers as discussed above. The biomolecules may further possess a detectable label, such as a luminescent, fluorescent or radioactive label, and/or an affinity tag.

In one embodiment of the present invention, the substrate may be used as a sample holder. Exemplary substrates may include microtiter strips or plates. In one specific embodiment, a sample such as a diluted serum may be pipetted into the wells of the microtiter plate or strip. A binding between the biomarkers in the serum and the biomolecules takes place. The presence or absence of the specific biomarkers or a combination of biomarkers as discussed above may indicate the infection status of the mammal.

The kit may further include a means of detection. The means of detection may include any detection method as discussed above. In one embodiment, the means of detection may be a spectroscopic technique, such as UV-Vis or MS. In one specific embodiment, the means of detection may be ELISA.

In one embodiment, the kit may include standard data for specific biomarker or a combination of biomarkers as discussed. One may compare the test result of a mammalian sample with the standard data for specific biomarker or a combination of biomarkers to determine the infection status of the mammal. For example, specific biomarkers or a combination of biomarkers may be visualized by a simple means of detection such as different colors. The detection result (e.g., showing one specific color) of a mammalian sample may be compared with the standard data (e.g., different colors for different biomarkers) to determine the infection status of the mammals.

In one embodiment, the kit may also be in the form of reagents (e.g. protein extract) that can be inoculated into animals to estimate the level of cell-mediate immunity (e.g. single intradermal comparative skin test, SICST). The reagents may include any of the biomarkers as discussed above. In one embodiment, the reagents may also include any genetically engineered vaccines. Suitable genetically engineered vaccines may include those Applicants previously proposed in PCT patent application publication WO20141640055A1, which is incorporated herein in its entirety.

The diagnostic kit may also include one or more of the following: instructions for use (detailing the method of the first aspect of the invention); sample collection apparatus (such as a needle and syringe); a chart for interpretation of the results; an electronic readout system; software providing a database for accurate data management.

The present invention has been described in terms of one or more preferred embodiments, and it should be appreciated that many equivalents, alternatives, variations, and modifications, aside from those expressly stated, are possible and within the scope of the invention.

### EXAMPLES

### Example 1—Proteomic Analysis of M. avium Subsp. paratuberculosis Vaccine Candidates

Johne's disease (JD) is a worldwide health problem for dairy herds that carries a heavy economic burden for producing safe food. Infected cattle suffer from chronic diarrhea, weight loss, low milk yield and low, but persistent mortality (1). For the dairy industry alone, the economic losses caused by JD are estimated to range between $200-$500 million annually, in the USA alone (2, 3). Identifying protective vaccine candidates against JD could be the cornerstone of controlling this widespread infection. In our group, we deciphered genomic information available for M. ap to identify key gene regulators that could control the expression of large number of genes. Throughout the genome of M. ap there are 19 sigma factors that act as global gene regulators that could contribute to the ability of M. ap to grow in many environments (4). Through previous funding from USDA, we examined several M. ap sigma (σ) factors that were important for growth in murine macrophages. Using transcriptional profiling, we compared mid-log phase M. ap to M. ap that had infected IFN-γ activated macrophages for 2 and 24 hours. Of the 19 sigma factors monitored, 6 sigma factor transcripts were up-regulated and one sigma factor transcript was down-regulated during the 24 hour time frame. Of the up-regulated transcripts, the sigL transcript was the only transcript up-regulated 2 hours after infection while sigH was up-regulated at 24 hrs (5). SigL is implicated in cell membrane protein biosynthesis as well as virulence in M. tuberculosis (6) while SigH was shown to be involved in combating the host intracellular responses such as oxidative stress (7).

To assess the role of sigL and sigH in M. ap virulence, we replaced the target sigma factors gene coding regions with a hygromycin-resistant gene cassette in M. ap K-10 using a specialized transduction protocol that was adapted for M. ap. Both genes were shown to be necessary for M. ap virulence in different stages of murine infection as detailed before (5, 8). Interestingly, the same mutants were shown to provide protective immunity against challenge with the virulent strain of M. ap when they were used as vaccine candidates in mice. To better analyze proteins expressed in each mutant, we grow cultures of M. apΔsigL, M. apΔsigH mutants and the wild type parent strain, M. ap K10 to mid-log phase. All cultures were washed twice in PBS, resuspended in buffer cocktail with endonuclease before proteomic analysis using nano-Liquid Chromatography-Mass Spectroscopy-MS (nano-LC MS/MS) at the University of Wisconsin Biotechnology Center. From 3 biological replicates, a total of ˜900 proteins were identified in this analysis comparing sigL and sigH mutant to M. ap K10 proteome.

### Diagnostic Markers for JD-Vaccinated Animals

A major problem in employing mass vaccination program for the control of JD in dairy herds is the inability to differentiate between infected and vaccinated animals with the current vaccine (DIVA principal). In addition, vaccinated animals could not be differentiated from positive reaction to the infection with M. bovis, a significant health problem for domesticated and wildlife animals. However, the DIVA principal and ability to distinguish between M. bovis and JD vaccinated animals could be achieved in genetically engineered vaccines (such as live attenuated vaccines based on sigL and sigH mutant) using a novel approach designed by the Applicant. In this approach, a simple blood test targeting proteins or sequences present in M. ap wild type strain and with lower expression level in the vaccine strain or even not encoded in the M. bovis genome would be developed. The target proteins include the following list of genes that could be used for the sigL-based vaccines.

TABLE 5
M. ap proteins that are significantly over-expressed
in the wild type strain and not in the sigL-vaccine
and could be used for sigL-DIVA testing.

| | Accession | Fold Change | |
|---|---|---|---|
| Number | Number | (K10/sigH) | Name/Function |
| 1 | Q73SF4 | 1.75 | hypothetical protein |
| 2 | Q73Y73 | 2.66 | aldehyde dehydrogenase (NAD+) |
| 3 | Q73ZE6 | 2.13 | nucleotide-sugar epimerase EpiA |
| 4 | Q73SL7 | 2.69 | hypothetical protein Mb0574c |
| 5 | Q73VK6 | 1.14 | oxidoreductase |
| 6 | Q73XZ0 | 1.88 | antigen CFP2 |
| 7 | Q740D1 | 4.71 | peptide synthetase Nrp |
| 8 | Q73UE0 | 1.99 | cutinase |

TABLE 6
M. ap proteins that are significantly over-expressed
in the wild type strain and not in the sigH-vaccine
and could be used for sigH-DIVA testing.

| | Accession | Fold Change | |
|---|---|---|---|
| Number | Number | (WT/sigH) | Name/Function |
| 1 | Q73VL6 | 3.05 | diguanylate cyclase (GGDEF) |
| | | | domain-containing protein |
| 2 | Q73YW9 | 1.64 | PE family protein, partial |
| 3 | Q741L4 | 1.88 | hypothetical protein |
| 4 | Q744E5 | 2.67 | ABC transporter ATPase |
| 5 | Q73YP5 | 2.47 | Pup--protein ligase |
| 6 | Q73WE5 | 1.78 | arginine decarboxylase |
| 7 | Q73U21 | 1.88 | PE family protein PE17 |
| 8 | Q73UH9 | 2.16 | XRE family transcriptional |
| | | | regulator |
| 9 | Q741M5 | 2.11 | nitroreductase |
| 10 | Q742F4 | 2.72 | metallo-beta-lactamase |
| 11 | Q73SU6 | 2.47 | 3-ketoacyl-ACP reductase |

In addition, another vaccine candidate is based on lipN mutant. In this case, epitopes that are different in the M. ap protein compared to those in M. bovis will be the target for DIVA diagnostic test.

FIG. 1 shows the alignment plot of amino acids deduced from the protein sequence in LipN of both M. paratuberculosis and M. bovis. Peptides conserved in M. paratuberculosis sequence but absent from M. bovis would be the target for developing the DIVA test.

### Example 2—Biomarkers for Early Diagnosis and Differentiation of Mycobacterial Infections

Johne's disease, caused by Mycobacterium avium subspecies paratuberculosis (MAP) is a chronic gastroenteritis of ruminants. Although infection often occurs within the first few months of life, clinical signs do not appear until 2-5 years of age. Current diagnostic tests, such as fecal culture and ELISA, have poor sensitivity for detection of the sub-clinical phase of disease. Therefore, biomarkers have been increasingly investigated as a method for sub-clinical detection.

In this project, we set out to develop rapid assays (e.g. PCR or field skin test) for early detection of presence of Johne's disease and for the differentiation of Johne's disease vaccinated vs. infected animals (with MAP or M. bovis). To speed up the project outcome, we capitalized on ongoing vaccine study in goats (Capra hircus) and collected Peripheral blood mononuclear cells (PBMC's) for transcriptional profiling followed by gene prediction for disease initiation and progression.

The PBMC's have been shown to be a predictor of infection and inflammatory disease. The PBMC transcriptomes of the goats were profiled using RNA-sequencing (RNA-Seq) to evaluate differential gene expression between a subset of samples from either 30 days post-vaccination, 30 days post-infection, or a naive, non-infected control group (3-4 biological replicates per group). Preliminary results on differential gene expression indicated the presence of 88 significantly differentially expressed genes out of 11,009 genes between goats at 30 days post-infection and the naïve, non-infected controls. The 30 days post-vaccination group had 720 out of 10,985 and 746 out of 11,099 significantly differentially expressed genes compared to the naïve, non-infected control group and the 30 days post-infection group, respectively. However, preliminary evaluation of the expressed genes indicated a large number of genes with immunological and inflammatory functions, including IL-18 binding protein, IFN-γ, IL-17A, and IL-22. Because of this inquiry, Table 7 summarizes selected genes/targets suitable to use in the present invention.

TABLE 7
List of DNA markers that are derived from the host transcriptome analysis and can be used
for early diagnosis of Johne's disease in ruminants (cattle, goats, sheep and camels).

| | | | | | |
|---|---|---|---|---|---|
| | | | | Homolog in | |
| Locus in goats | Symbol | Protein | Description | Bos taurus (cows) | Homolog description |

Selected list of host (goat and cow) markers that can differentiate infected from naïve animals.

| | | | | | |
|---|---|---|---|---|---|
| NW_005125111.1: 0-184 | unplaced genomic | N/A | N/A | | |
| | scaffold | | | | |
| NW_005101181.1: | unplaced genomic | N/A | N/A | | |
| 1703-1858 | scaffold | | | | |
| NW_005101181.1: | LOC102180841 | XP_005701370.1 | PREDICTED: multidrug | XP_005199610 | multidrug resistance- |
| 168292-168418 | | | resistance-associated | | associated protein 4- |
| | | | protein 4-like | | like isoform X1 |
| NC_022320.1: | Non-coding | N/A | N/A | | |
| 39973839-39974080 | region | | | | |
| NC_022297.1: | IL-22 | XP_005680263.1 | interleukin 22 | NP_001091849.1 | interleukin 22 |
| 44037534-44043184 | | | | | |
| NC_022296.1: | Non-coding | N/A | N/A | | |
| 81262820-81263390 | region | | | | |
| NW_005101844.1: | ABCC4 | XP_005701761.1 | PREDICTED: multidrug | XP_010820300.1 | PREDICTED: multidrug |
| 141791-142987 | | | resistance-associated | | resistance-associated |
| | | | protein 4-like, partial | | protein 4 isoform X1 |
| NW_005101711.1: | LOC102185556 | XP_005701708.1 | PREDICTED: multidrug | XP_003585348.3 | PREDICTED: multidrug |
| 48628-48757 | | | resistance-associated | | resistance-associated |
| | | | protein 4-like | | protein 4 isoform X1 |
| NW_005132660.1: 0-240 | unplaced genomic | N/A | N/A | | |
| | scaffold | | | | |
| NW_005109943.1: 2-224 | unplaced genomic | N/A | N/A | | |
| | scaffold | | | | |
| NW_005149706.1: 0-366 | unplaced genomic | N/A | N/A | | |
| | scaffold | | | | |
| NW_005153011.1: 2-407 | unplaced genomic | N/A | N/A | | |
| | scaffold | | | | |
| NW_017189548.1: | LOC108634521 | N/A | ncRNA | N/A | N/A |
| 2899 . . . 17746 | | | | | |
| NC_030819.1: | LOC108637251 | N/A | N/A | N/A | multidrug resistance- |
| complement | | | | | associated protein 4-like |
| (13836329 . . . 13914672) | | | | | |
| NC_030819.1: | LOC108637252 | N/A | N/A | N/A | multidrug resistance- |
| 13926013 . . . 14000960 | | | | | associated protein 4-like |
| NW_017189646.1: | LOC108634594 | N/A | N/A | N/A | multidrug resistance- |
| complement | | | | | associated protein 4-like |
| (5337 . . . 40350) | | | | | |
| NC_030824.1: | FAM198B | N/A | N/A | N/A | family with sequence |
| complement | | | | | similarity 198 member B |
| (30222726 . . . 30294233) | | | | | |
| NC_030822.1: | LOC108637671 | N/A | N/A | N/A | tripartite |
| 34764774 . . . 34772382 | | | | | motif-containing |
| | | | | | protein 5-like |
| NC_030829.1: | CDCP1 | 102187276 | CUB domain containing | XP_002697164; | |
| 54084869 . . . 54144689 | | | protein | XP_612363 | |
| NC_030812.1: | TMTC1 | 102185637 | transmembrane and | N/A | |
| 78337529 . . . 78655742 | | | tetratricopeptide repeat | | |
| NC_030832.1: | BAIAP2L1 | 102173150 | BAI1 associated | XP_003584109; | |
| 38530654 . . . 38607511 | | | protein 2 like | XM_003584061; | |
| | | | | XP_003587892; | |
| | | | | XM_003587844 | |
| NC_030812.1: | MEI1 | 102169168 | meiotic double-stranded | NP_001295589 | |
| 111693222 . . . 111748937 | | | break formation protein | | |
| NC_030818.1: | SEPT10 | 102171885 | septin 10 | NP_001039641 | |
| 43871666 . . . 43923217 | | | | | |
| NC_030812.1: | IFNG | 100860815 | interferon gamma | NP_776511 | |
| 44984285 . . . 44988400 | | | | | |
| NC_030830.1: | IL17F | 102171111 | interleukin 17F | NP_001179011 | |
| 24511444 . . . 24519042 | | | | | |
| NC_030814.1: | FCER2 | 102171507 | Fc fragment of IgE | N/A | |
| 93891943 . . . 93902156 | | | receptor II | | |
| NC_030825.1: | ADGRG1 | 102171366 | G protein-coupled | NP_001077125 | |
| 26945415 . . . 26990225 | | | receptor G | | |
| NC_030822.1: | APBB1 | 102179305 | amyloid beta precursor | NP_001068654 | |
| complement | | | protein binding family | | |
| (35694810 . . . 35718058) | | | B member | | |
| NC_030815.1: | PIWIL2 | 102173845 | piwi like RNA-mediated | XP_015320079; | |
| 69261357 . . . 69324811 | | | gene silencing | XM_015464593; | |
| | | | | XP_015328077; | |
| | | | | XM_015472591 | |

Selected list of host (goat and cow) markers that can differentiate live attenuated vaccinated (LAV) from naïve animals.

| | | | | | |
|---|---|---|---|---|---|
| NC_022296.1: | Non-coding | N/A | N/A | | |
| 32351255-32351413 | region | | | | |
| NC_022307.1: | Non-coding | N/A | N/A | | |
| 44045143-44403012 | region | | | | |
| NC_022295.1: | Non-coding | N/A | N/A | | |
| 13176472-13182094 | region | | | | |
| NC_022321.1: | Non-coding | N/A | N/A | | |
| 6000551-6000875 | region | | | | |
| NW_005126018.1: 16-203 | unplaced genomic | N/A | N/A | | |
| | scaffold | | | | |
| NW_005101711.1: | LOC102185556 | XP_005701708.1 | PREDICTED: multidrug | XP_003585348.3 | PREDICTED: multidrug |
| 48628-48757 | | | resistance-associated | | resistance-associated |
| | | | protein 4-like | | protein 4 isoform X1 |
| NW_005101844.1: | ABCC4 | XP_005701761.1 | PREDICTED: multidrug | XP_010820300.1 | PREDICTED: multidrug |
| 141790-142987 | | | resistance-associated | | resistance-associated |
| | | | protein 4-like, partial | | protein 4 isoform X1 |
| NW_005101645.1: | unplaced genomic | N/A | N/A | | |
| 16151-23647 | scaffold | | | | |
| NW_017189548.1: | LOC108634521 | 108634521 | ncRNA | | |
| 2899 . . . 17746 | | | | | |
| NC_030826.1: | NOS2 | 100860742 | nitric oxide synthase 2 | NP_001070267 | |
| complement | | | | | |
| (19203362 . . . 19245850) | | | | | |
| NC_030819.1: | LOC108637251 | 108637251 | multidrug resistance- | | |
| complement | | | associated protein 4-like | | |
| (13836329 . . . 13914672) | | | | | |
| NC_030809.1: | TINAGL1 | 102169636 | tubulointerstitial | XP_015315454; | |
| 13806906 . . . 13817913 | | | nephritis antigen like | XP_015317919; | |
| | | | | XP_015315453; | |
| | | | | XM_015459967; | |
| | | | | XP_015317918; | |
| | | | | XM_015462432 | |
| NW_017189666.1: | RETN | 102170965 | resistin | NP_899206 | |
| complement | | | | | |
| (10281 . . . 11678) | | | | | |
| NC_030809.1: | C1QL2 | 102176742 | complement C1q like 2 | NP_001192765 | |
| 65236017 . . . 65240282 | | | | | |
| NC_030810.1: | TDRD10 | 102174259 | tudor domain containing 10 | XP_005197751; | |
| 104057279 . . . 104111513 | | | | XM_005197694; | |
| | | | | XP_005203865; | |
| | | | | XM_005203808 | |
| NC_030817.1: | TGFB3 | 102189962 | transforming growth | NP_001094653; | |
| 15809952 . . . 15835252 | | | factor beta 3 | XP_005212263; | |
| | | | | XP_005212264 | |
| NC_030814.1: | ADGRE2 | 102171592 | adhesion G protein-coupled | | |
| 96350745 . . . 96400437 | | | receptor E2 | | |
| NC_030831.1: | LIPG | 102191574 | lipase G endothelial type | | |
| 49397175 . . . 49422345 | | | | | |
| NC_030826.1: | KCNJ2 | 102168940 | potassium voltage-gated | NP_776798 | |
| complement | | | channel subfamily J member 2 | | |
| (59803404 . . . 59814242) | | | | | |
| NC_030817.1: | AQP9 | 102181396 | aquaporin 9 | XP_015328629; | |
| 51022717 . . . 51074079 | | | | XM_015473143; | |
| | | | | XP_015328630; | |
| | | | | XM_015473144 | |
| NC_030820.1: | BPI | 102185756 | bactericidal/permeability- | NP_776320 | |
| 66719462 . . . 66768979 | | | increasing protein | | |
| NC_030814.1: | IL9 | 102179848 | interleukin 9 | XP_015319783; | |
| 63194434 . . . 63197324 | | | | XM_015464297; | |
| | | | | XP_015327708; | |
| | | | | XM_015472222 | |
| NC_030818.1: | IL1R2 | 102186601 | interleukin 1 receptor | NP_001039675; | |
| 6611732 . . . 6648492 | | | type 2 | XP_010808117; | |
| | | | | XP_010808118 | |
| NC_030818.1: | IL36B | 102182235 | interleukin 36 beta | XP_002691396; | |
| 46359268 . . . 46368303 | | | | XM_002691350; | |
| | | | | XP_002700827; | |
| | | | | XM_002700781 | |
| NC_030812.1: | IGF1 | 100860838 | insulin like growth factor | XP_005206547; | |
| complement | | | | XP_015326547; | |
| (64862983 . . . 64943172) | | | | XP_015326549 | |
| NW_017190169.1: | BGN | 102183219 | biglycan | NP_847888; | |
| complement | | | | XP_005227715 | |
| (56783 . . . 70690) | | | | | |
| NC_030815.1: | PIWIL2 | 102173845 | piwi like RNA-mediated | XP_015320079; | |
| 69261357 . . . 69324811 | | | gene silencing | XM_015464593; | |
| | | | | XP_015328077; | |
| | | | | XM_015472591 | |
| NC_030816.1: | RAET1E | 108636743 | retinoic acid early | | |
| 74364855 . . . 74372073 | | | transcript | | |
| NC_030810.1: | CRABP2 | 102174348 | cellular retinoic acid | NP_001008670 | |
| complement | | | binding protein 2 | | |
| (105983749 . . . 105989432) | | | | | |

Selected list of host (goat and cow) markers that can differentiate inactivated-vaccine immunized from naïve animals.

| | | | | | |
|---|---|---|---|---|---|
| NW_005125111.1: 0-184 | unplaced genomic | N/A | N/A | | |
| | scaffold | | | | |
| NC_022320.1: | Non-coding | N/A | N/A | | |
| 39973839-39974080 | region | | | | |
| NC_022303.1: | Non-coding | N/A | N/A | | |
| 46207878-46237242 | region | | | | |
| NC_022296.1: | Non-coding | N/A | N/A | | |
| 81262820-81263390 | region | | | | |
| NW_005101711.1: | LOC102185556 | XP_005701708.1 | PREDICTED: multidrug | XP_003585348.3 | PREDICTED: multidrug |
| 48628-48757 | | | resistance-associated | | resistance-associated |
| | | | protein 4-like | | protein 4 isoform X1 |
| NW_005102056.1: | LOC102190036 | XP_005701827.1 | PREDICTED: tyrosine-protein | NP_786982.1 | tyrosine-protein |
| 2049-9786 | | | phosphatase non-receptor | | phosphatase non-receptor |
| | | | type substrate 1-like, partial | | type substrate 1 |
| | | | | | precursor |
| NC_022309.1: | — | XP_005691363.1 | PREDICTED: protein FAM198B | NP_001077247.1 | protein FAM198B |
| 40520580-40588889 | | | | | |
| NW_005101931.1: | LOC102180487 | XP_005701808.1 | PREDICTED: interferon | NP_001069925.2 | uncharacterized protein |
| 48185-54192 | | | alpha-inducible protein | | LOC617420 |
| | | | 27-like protein 2-like | | |
| NW_005164924.1: 1-636 | unplaced genomic | N/A | N/A | | |
| | scaffold | | | | |

Selected list of host (goat and cow) markers that can differentiate LAV-vaccine immunized from infected animals.

| | | | | |
|---|---|---|---|---|
| NC_030826.1: | LOC106503226 | 106503226 | | |
| 39149778 . . . 39151507 | | | | |
| NC_030826.1: | PMP22 | 102184371 | peripheral myelin | NP_001094626; |
| complement | | | protein 22 | XP_005220437; |
| (32435859 . . . 32463764) | | | | XP_010814341 |
| NC_030822. 1: | ART5 | 102169686 | ADP-ribosyl- | |
| complement | | | transferase 5 | |
| (31912716 . . . 31916296) | | | | |
| NC_030822.1: | LOC102169116 | 102169116 | ecto-ADP-ribosyl- | |
| complement | | | transferase 5 | |
| (31947901 . . . 31951735) | | | | |
| NC_030818.1: | GNLY | 102191341 | granulysin | NP_001068611 |
| complement | | | | |
| (48786756 . . . 48789216) | | | | |
| NC_030809.1: | ASAP3 | 102182646 | ArfGAP with SH3 domain | NP_001076915 |
| complement | | | ankyrin repeat and PH | |
| (6533315 . . . 6583406) | | | domain 3 | |
| NC_030828.1: | LOC108633178 | 108633178 | granzyme B-like | |
| complement | | | | |
| (69374237 . . . 69377734) | | | | |
| NC_030826.1: | TBKBP1 | 102172659 | TBK1 binding protein | XP_001253301; |
| complement | | | | XP_005195770; |
| (38379103 . . . 38396803) | | | | XP_005220704; |
| | | | | XP_010814497; |
| | | | | XP_010822429; |
| | | | | XP_015314281 |
| NC_030825.1: | SLC17A7 | 102169042 | solute carrier family 17 | NP_001091515 |
| complement | | | member 7 | |
| (56899764 . . . 56911150) | | | | |
| NC_030826.1: | LOC108638192 | 108638192 | | |
| 53112179 . . . 53116348 | | | | |
| NC_030812.1: | IFNG | 100860815 | interferon gamma | NP_776511 |
| 44984285 . . . 44988400 | | | | |

### Materials and Methods

Animals—Approximately one week-old kids were purchased from a farm with no previous history of Johne's disease. All study kids, and their dams, tested negative for M. paratuberculosis by ELISA for serum antibody (Paracheck®, Biocor Animal Health, Omaha, NE). Additionally, fecal samples collected from the originating farm environment were negative for M. paratuberculosis by culture. All kids were housed in a restricted biosafety animal facility (BSL-2). All animal care was handled in accordance to the standards of the University of Wisconsin-Madison Animal Care and Use Committee. The kids were randomly assigned to one of four groups as shown in Table 8. One group of kids (n=6 but only 4 used for transcriptome analysis) were vaccinated with a live-attenuated vaccine (LAV) construct (M. paratuberculosis ΔlipN mutant (Wu et al., 2007)) at a dose of 1×10⁹ CFU/animal. The second groups of kids (n=4) were vaccinated with the USDA-licensed inactivated vaccine (Mycopar®). A third group inoculated with PBS served as the vaccine control. Both vaccines and PBS were given subcutaneously. At 60 days post-vaccination, kids in these three groups were inoculated with M. paratuberculosis strain JTC1285 at a dose of 1×10⁸ CFU administered orally in the milk replacer for three consecutive days. A fourth group (n=4), inoculated with PBS and not challenged with M. paratuberculosis served as a naïve control. Goat kids were monitored daily for signs of clinical disease and evaluated monthly for potential weight loss. A detailed report on the outcome of this vaccine/challenge study was previously published (Shippy et al., 2017).

TABLE 8
Experimental Groups

| | | | Vaccine | |
|---|---|---|---|---|
| Group | No. | Vaccine* | Dose | Challenge Strain/Dose** |
| Infected | 4 | PBS | 0.5 ml | M. paratuberculosis JTC1285/ |
| | | | | 1 × 10⁸ CFU |
| LAV- | 4 | M. ap | 1 × 10⁹ | M. paratuberculosis JTC1285/ |
| vaccinated | | ΔlipN | CFU | 1× 10⁸ CFU |
| Mycopar- | 3 | Mycopar ® | 0.5 ml | M. paratuberculosis JTC1285/ |
| vaccinated | | | | 1 × 10⁸ CFU |
| Naïve | 4 | PBS | 0.5 ml | None |
| Control | | | | |

*All vaccines were given subcutaneously.
**Challenge dose was given orally in milk replacer for three consecutive days and was performed at 60 days post-vaccination in LAV- and Mycopar- vaccinated groups.

Isolation of blood cells—Blood samples (10 ml) were collected from the jugular vein of goats into EDTA vacutainer tubes pre-vaccination, 1 week, 30 days, 60 days post-vaccination and 1 week post-challenge (for 3 groups), and then monthly for 12 months. Peripheral blood mononuclear cells (PBMC) were isolated using Histopaque®-1077 (Sigma-Aldrich®) with the following modifications. Anti-coagulated blood was diluted with an equal volume of RPMI-1640 medium (Sigma Aldrich®), layered over 10 ml of Histopaque®-1077, and centrifuged at 400×g for 30 minutes at room temperature. Following centrifugation, PBMC's were aspirated from the interface and washed twice with RPMI-1640 medium. Residual red blood cells were lysed with 0.83% NH₄Cl₂. The PBMC's were then resuspended in complete culture medium (RPMI-1640 containing 10% fetal bovine serum, 1% L-glutamine, 1% penicillin/streptomycin (final concentration 100 IU/ml), and 1% nonessential amino acids). Cell density was determined by use of 0.4% Trypan blue stain and a hemocytometer.

PBMC stimulation and RNA extraction—PBMC's were plated at a density of 1×10⁶/well in 96 well plates with either medium alone (non-stimulated) or M. paratuberculosis whole cell lysate (WCL). The WCL was prepared by resuspending the centrifuged cell pellet of actively grown M. paratuberculosis (O.D. ˜1.0) in protein lysis buffer (100 mM Tris-Cl, 100 mM NaCl, 5 mM MgCl₂, 1 mM PMSF, complete ultra-protease inhibitor cocktail (Roche, Indianapolis, IN; pH 7.5) and bead-beating to homogenize (maximum pulse for 45 sec for a total of 4 pulses; with cooling on on ice for 30 sec between pulses). The supernatant was then transferred to a new 1.5 ml tube and non-soluble material was removed by centrifugation at 10,000×g for 5 min at 4° C. The protein content of the supernatant was measured via the Pierce™. BCA protein assay (Thermo Fisher Scientific), aliquoted and stored at −80° C. until used. Final concentrations of WCL was 10 μg/ml. IL-2 was added to all wells at a concentration of 100 U/ml. Plates were incubated at 37° C. with 5% CO₂ for 24 hours. Supernatants were then removed and cell pellets were stored in 100 μl TRIzol® and frozen at −80° C. until used for RNA extraction. RNA was extracted from stimulated PBMC's using TRIzol® and RNeasy® Mini Kit (Qiagen®) according to manufacturer's directions for the remainder of the extraction. TURBO DNA-free™ DNase Treatment (Ambion®) was used to eliminate residual genomic DNA. RNA quantity and quality was assessed using the RNA Pico Series Chip on the Bioanalyzer 2100 (Agilent). RNA integrity numbers (RINs) >8 were obtained for all total RNA samples purified.

RNA Sequence Analysis-RNA-Sequencing (RNA-Seq) was performed by the University of Wisconsin-Madison Biotechnology Center on RNA extracted from WCL-stimulated PBMC's from goats at 30 days post-vaccination, 30 days post-challenge (PBS vaccinated), or at the same time for the naïve control group (4 goats/biological replicates per group). A total of 1 μg of RNA was used as input for TruSeq® RNA Sample Prep Rev. F (March 2014; Illumina®). Paired-end RNA Sequencing was performed on the Illumina HiSeq 2000 sequencer according to manufacturer's instructions.

Raw RNA-Seq reads were uploaded to CLC Genomics Workbench 8.5 (Qiagen, Redwood City, CA) for processing. Two read files from one RNA sample were paired and trimmed. The ambiguous trim limit was set at 1 and quality trim limit was at 0.05. Reads shorter than 25 nucleotides were excluded. The trimmed sequences were then mapped to the reference genome sequence of Capra hircus assembly ARS1 (Bickhart et al., 2017) and read counts against the reference genome annotation tracks, generated with files, available at ncbi.nlm.nih.gov/genomes/Capra_hircus, were compiled and tabulated using the CLC Genomics Workbench NGS tools. The mapping parameters were set as follows: mismatch cost, 2; insertion and deletion cost, 3; length and similarity fraction, 0.8. Unique gene reads from each sample were exported from CLC Genomics Workbench and used for normalization and differential gene expression analysis with an R package, DESeq2 version 1.16.1 (Love et al., 2014). Transcripts that had an average of normalized read count <3 in all three tested groups were excluded from the analysis (N=11,541). Differentially expressed transcripts are defined as transcripts with fold changes ≥2.0 or ≤−2.0 (or Loge-transformed fold changes ≥1.0 or ≤−1.0), and p-value <0.05 when compared to the naïve control group.

Gene ontology (GO) analysis was performed for the differentially expressed genes with agriGO, an automated tool to identify enriched GO terms, which is specially focused on agricultural species (Du et al., 2010). The gene products are categorized with respect to biological processes, cellular components, and molecular functions. Because the gene ontology in the goat genome is poorly annotated, we chose the Bos taurus ENSEMBL genome B2G list (2010 version) as the reference genome. Goat genes (assembly ARS1) with an Entrez gene name were mapped to the counterparts in the bovine genome, resulting in a total gene list of 9,115 GO-annotated genes. Goat DE genes identified in the RNA-Seq analysis were also mapped to the bovine genome and used as query lists against the 9,115-gene reference. FDR was calculated using the Fisher test.

Network analysis was performed using the STRING database (Szklarczyk et al., 2015) with DE transcripts identified in this study. The input DE transcripts were treated as homologues of Bos taurus because of availability in the database.

Quantitative RT-PCR-cDNA was synthesized from each RNA sample using SuperScript III Reverse Transcriptase (Invitrogen, Waltham, MA) and oligo(dT)₁₂₋₁₈ Primer according to manufacturer's instructions. Quantitative PCR (qPCR) assays were performed in triplicates for each cDNA sample. Primers were designed across adjacent exons in order to differentiate products from genomic DNA and cDNA. The GAPDH gene served as an internal control to normalize the data for the ΔΔCt relative quantitation method. The assays were performed on an Applied Biosystems StepOne Plus Real-Time PCR System (Foster City, CA), and the cDNA amplifications were monitored by the measurement of SYBR Green fluorescence at a specific cycle threshold. Each reaction was carried out in a 20 μl volume that contained 10 μl of 2×GoTaq qPCR Master Mix (Promega, Madison, WI), 5.0 μl of ddH2O, 0.5 μl of each primer (10 μm) and 4.0 μl of the template (100-150 ng/ul). The qPCR amplification process began with the temperature at 95° C. for 2 min, followed by 40 cycles of the amplification process (95° C. for 3 s, 60° C. for 30 s). Subsequent to the cycling process, melting curves were generated by inclining the temperature from 60° C. to 95° C. at 0.3° C./s increments. With the exception of the infected group at 1 month post-challenge where two samples were used, cDNA samples from three animals in each group were included in the qPCR analysis. Average ΔΔCt values and standard errors of the mean (SEM) of the three measurements were calculated and transformed to linear fold change.

qRT-PCR primers

| | | | SEQ |
|---|---|---|---|
| | Primer | | ID |
| Primer ID | name | Primer sequence | NO: |
| AMT2341 | SEPT10_F | ggtgagcgccagaggaa | 4 |
| AMT2342 | SEPT10_R | cagcttctcctcttggtggac | 5 |
| AMT2343 | IL18BP_F | aactggatcccagacccc | 6 |
| AMT2344 | IL18BP_R | gtagctgctgggagcgc | 7 |
| AMT2351 | IL17A_F | ggaacacgaactccagaaggc | 8 |
| AMT2352 | IL17A_R | acagagttcatgtgatggtccac | 9 |
| AMT2353 | CRABP2_F | accaccgtgcgtaccac | 10 |
| AMT2354 | CRABP2_R | ggaggtcttgggaccctctc | 11 |
| AMT2355 | IL36_F | cgttaatagcagttccttctagcaac | 12 |
| AMT2356 | IL36_R | ggatagccctggatttctgtgc | 13 |
| AMT2361 | RETN_F | tgaggcagtaaggaacattggc | 14 |
| AMT2362 | RETN_R | agtccatgcctgcgcac | 15 |
| AMT2363 | IFNG_F | gcagctctgagaaactggagg | 16 |
| AMT2364 | IFNG_R | tccggcctcgaaagagattct | 17 |
| AMT2365 | GAPDH_F | ggcgtgaaccacgagaagtataa | 18 |
| AMT2366 | GAPDH_R | ggcagtgatggcgtggac | 19 |
| AMT 2899 | ABCC4_F | cttggatcgccatacccctc | 20 |
| AMT 2900 | ABCC4_R | gggctccgggttgtagattc | 21 |
| AMT 2914 | IL 17F_F | gaggaccacattgtgagggt | 22 |
| AMT 2915 | IL 17F_R | cgggtgatgttgtaatcccag | 23 |
| AMT 2918 | TINAGL1_F | cgacgaggggttgtgtctg | 24 |
| AMT 2919 | TINAGL1_R | acatagctattggggcagcg | 25 |
| AMT 2971 | FAM198B_F | tcatccaagatggccgcc | 26 |
| AMT 2972 | FAM198B_R | gccagcacttctgtttcagc | 27 |
| AMT 2973 | AOAH_F | gaaatcacggaggagtggca | 28 |
| AMT 2974 | AOAH_R | aacagctgtgaaaccacctca | 29 |
| AMT 2988 | IL 22_F | cagggaatcaatcaggtgacga | 30 |
| AMT 2989 | IL 22_R | atgggggtggaattcatcgg | 31 |
| AMT 2992 | MEI1_F | cagtgaagtgctcgtctggt | 32 |
| AMT 2993 | MEI1_R | cgactcaatcccatacaccgt | 33 |
| AMT 2994 | CDCP1_F | aagccaagcttccgctatca | 34 |
| AMT 2995 | CDCP1_R | cgatgacagtcaggtccgtg | 35 |

### Results

Transcriptome analysis of goat groups. The transcriptome analysis of goats infected with M. paratuberculosis and/or vaccinated LAV vaccine strain M. ap ΔlipN is a proportion of a larger study that examined the performance of this vaccine published earlier (Shippy et al., 2017). The transcriptome analysis is the focus of this report. The summary statistics of the RNA-Seq data for each replicate are shown in Table 9. Mean values of 58.88 million raw reads were generated per library (each RNA sample). Following trimming of reads based on read length, quality score and adapter sequences, an average of 20.04 million paired reads remained. Alignment of the trimmed RNA-Seq reads to the Capra hircus reference genome yielded mean values per library of 18.71 million paired reads (93.32%) mapped to unique locations.

TABLE 9
Summary statistics for Illumina RNA sequencing data

| | | | Total | % Total | | |
|---|---|---|---|---|---|---|
| | | Number of | Paired | Paired | | % |
| | Total | Read Pairs | Reads | Reads | Uniquely | Uniquely |
| Group/Replicate | Number of | Being | After | After | mapped | mapped |
| Number* | Reads | Trimmed | mapping | Trimming | reads | reads |
| Infected 1 | 73,105,634 | 22,810,292 | 25,147,671 | 68.8 | 23,533,313 | 93.58 |
| Infected 2 | 72,155,108 | 23,491,132 | 24,331,988 | 67.44 | 22,701,378 | 93.30 |
| Infected 3 | 68,005,382 | 21,295,316 | 23,355,033 | 68.69 | 21,941,420 | 93.95 |
| Infected 4 | 48,108,726 | 15,062,194 | 16,523,266 | 68.69 | 15,433,817 | 93.41 |
| LAV-vaccinated 1 | 73,973,058 | 24,360,882 | 24,806,088 | 67.07 | 23,253,486 | 93.74 |
| LAV-vaccinated 2 | 63,076,126 | 20,765,850 | 21,155,138 | 67.08 | 19,668,167 | 92.97 |
| LAV-vaccinated 3 | 34,967,370 | 11,169,558 | 11,898,906 | 68.06 | 11,062,093 | 92.97 |
| LAV-vaccinated 4 | 66,996,260 | 21,387,972 | 22,804,144 | 68.08 | 21,282,727 | 93.33 |
| Mycopar- | 60,074,726 | 20,141,682 | 19,966,522 | 66.47 | 18,707,059 | 93.69 |
| vaccinated 1 | | | | | | |
| Mycopar- | 70,284,036 | 23,645,792 | 23,319,122 | 66.36 | 21,978,596 | 94.25 |
| vaccinated 2 | | | | | | |
| Mycopar- | 64,746,832 | 21,633,920 | 21,556,456 | 66.59 | 20,141,993 | 93.44 |
| vaccinated 3 | | | | | | |
| Naive 1 | 73,575,076 | 22,859,182 | 25,357,947 | 68.93 | 23,681,558 | 93.39 |
| Naive 2 | 50,851,250 | 16,107,620 | 17,371,815 | 68.32 | 16,180,977 | 93.14 |
| Naive 3 | 46,913,012 | 15,083,746 | 15,914,633 | 67.85 | 14,815,443 | 93.09 |
| Naive 4 | 34,829,124 | 11,209,870 | 11,809,627 | 67.81 | 10,972,712 | 92.91 |

*Time when blood samples were taken: Infected: 30 days post-infection; LAV-vaccinated: 30 days post-LAV vaccination; Mycopar-vaccinated: 30 days post-Mycopar ® vaccination; Naïve:: 30 days post-PBS vaccination.

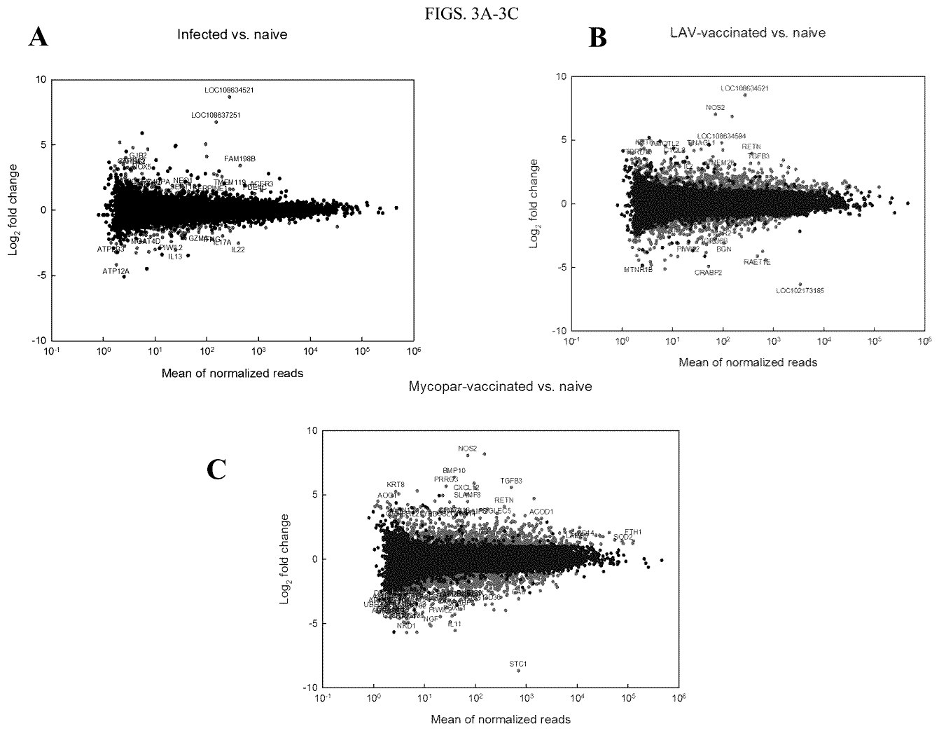

Changes in the goat transcriptomes related to infection or vaccination—Transcriptomes of different animal groups were analyzed to identify differentially expressed (DE) genes with significant change using a p-value threshold of ≥0.05 and ≥2-fold change. A summary of comparative numbers of differentially expressed genes is presented in Table 10. MA-plots in FIGS. 3A-3C depict the distributions of the DE transcripts PI and post-vaccination groups compared to naïve control group. Generally, the infected goat group had 226 significantly DE transcripts out of 17,380 (total goat transcripts identified by RNA-Seq) at 30 days PI in comparison to the naïve, non-infected controls. Of the 226 significantly DE transcripts, 113 were up-regulated in the PI group, while the other 113 were down-regulated. A total of 106 out of the 226 DE transcripts had more than a 2.8 fold change (or 1.5 log₂ fold change) with a selected group of known function listed in Table 11. On the other hand, the LAV-vaccinated goat group had 1018 significantly DE transcripts out of 17,380 compared to the naïve, non-infected control group. A total of 628 and 390 transcripts were up- and down-regulated, respectively. A total of 517 out of the 1018 had ≥2.8 fold change with a selected group of known function listed in Table 11. Additionally, when the transcripts of both LAV-vaccinated and infected groups were compared, at total of 1133 transcripts were significantly DE out of 17,380 (Table 10). Of these transcripts, 629 and 504 transcripts were up- and down-regulated, respectively. A total of 575 out of the 1133 DE transcripts were greater than a 2.8 fold change. Interestingly, the immunization with the inactivated, oil-based vaccine (Mycopar) triggered significant changes in a large number of goat genes (N=1714) including key genes involved in immune responses (Table 10).

TABLE 10
Differentially Expressed (DE) Genes for each comparison group

| Comparison | Total analyzed Genes | DE Genes* |
|---|---|---|
| Infected vs Naïve | 17,380 | 226 |
| LAV-vaccinated vs Naïve | | 1018 |
| Mycopar-vaccinated vs Naïve | | 1714 |
| LAV-vaccinated vs Infected | | 1133 |

*DE genes were identified as those with a p value threshold of ≤ 0.05

TABLE 11
Selected differentially up- or down-regulated genes by fold change,
between 30 days post-infection and naïve groups

| Gene symbol | Gene ID | Fold change | P value | Description |
|---|---|---|---|---|
| FAM198B | 102191727 | 10.70 | 0.0016 | family with sequence similarity 198 |
| | | | | member B |
| CDCP1 | 102187276 | 4.06 | 0.0143 | CUB domain containing protein 1 |
| TMTC1 | 102185637 | 3.63 | 0.0217 | transmembrane and tetratricopeptide |
| | | | | repeat containing 1 |
| BAIAP2L1 | 102173150 | 3.61 | 0.0196 | BAI1 associated protein 2 like 1 |
| MEI1 | 102169168 | 2.99 | 0.0155 | meiotic double-stranded break |
| | | | | formation protein 1 |
| SEPT10 | 102171885 | 2.41 | 0.0239 | septin 10 |
| IFNG | 100860815 | −3.36 | 0.0047 | interferon, gamma |
| IL17F | 102171111 | −2.33 | 0.0098 | interleukin 17F |
| FCER2 | 102171507 | −2.57 | 0.0001 | Fc fragment of IgE receptor II |
| ADGRG1 | 102171366 | −3.43 | 0.0037 | adhesion G protein-coupled receptor |
| | | | | G1 |
| APBB1 | 102179305 | −4.47 | 0.0002 | amyloid beta precursor protein |
| | | | | binding family B member 1 |
| PIWIL2 | 102173845 | −5.31 | 0.0400 | piwi like RNA-mediated gene |
| | | | | silencing 2 |

TABLE 12
Selected differentially up- or down-regulated genes by fold change,
between 30 days post-LAV-vaccination and naïve groups

| Gene symbol | Gene ID | Fold change | p-value | Description |
|---|---|---|---|---|
| NOS2 | 100860742 | 130.42 | 2.3E−09 | nitric oxide synthase 2 |
| TINAGL1 | 102169636 | 19.31 | 1.2E−05 | tubulointerstitial nephritis antigen like |
| RETN | 102176742 | 12.91 | 4.4E−13 | resistin |
| C1QL2 | 102176742 | 12.89 | 0.002 | complement C1q like 2 |
| TDRD10 | 102174259 | 11.54 | 0.019 | tudor domain containing 10 |
| TGFB3 | 102189962 | 9.13 | 0.0020 | transforming growth factor beta 3 |
| ADGRE2 | 102171592 | 5.90 | 0.0135 | adhesion G protein-coupled receptor E2 |
| LIPG | 102191574 | 5.28 | 0.0001 | lipase G, endothelial type |
| KCNJ2 | 102168940 | 4.82 | 0.0003 | potassium voltage-gated channel subfamily J member 2 |
| AQP9 | 102181396 | 4.72 | 0.0007 | aquaporin 9 |
| BPI | 102185756 | 3.61 | 0.0140 | bactericidal/permeability-increasing, protein |
| IL9 | 102179848 | −2.91 | 0.0083 | interleukin 9 |
| IL1R2 | 102186601 | −3.63 | 0.0055 | interleukin 1 receptor type 2 |
| IL36B | 102182235 | −5.46 | 0.0013 | interleukin 36 beta |
| IGF1 | 100860838 | −5.46 | 0.0463 | insulin, like, growth, factor, 1 |
| BGN | 102183219 | −8.57 | 0.0045 | biglycan |
| PIWIL2 | 102173845 | −8.57 | 0.009 | piwi like RNA-mediated gene silencing 2 |
| RAET1E | 108636743 | −17.27 | 0.0008 | retinoic acid early transcript 1E |
| CRABP2 | 102174348 | −30.12 | 2.0E−20 | cellular retinoic acid binding protein 2 |

TABLE 13
Selected differentially up- or down-regulated genes
by fold change, between 30 days post-
Mycopar ®-vaccination and naïve groups

| Gene symbol | Gene ID | Fold change | p-value | Description |
|---|---|---|---|---|
| NOS2 | 100860742 | 269.200 | 3.7E−11 | nitric oxide synthase 2 |
| BMP10 | 102185577 | 82.746 | 0.0003 | bone morphogenetic |
| | | | | protein 10 |
| TDRD10 | 102174259 | 18.438 | 0.0061 | tudor domain |
| | | | | containing 10 |
| RETN | 102170965 | 16.901 | 4.2E−12 | resistin |
| AMOTL2 | 102169708 | 14.389 | 0.0065 | angiomotin like 2 |
| KLRG2 | 102177407 | 12.733 | 2.9E−10 | killer cell lectin like |
| | | | | receptor G2 |
| IL21 | 100861248 | 8.124 | 4.6E−05 | interleukin 21 |
| C2 | 102176085 | 7.95 | 2.4E−7 | complement C2 |
| C3 | 100860826 | 6.495 | 0.0002 | complement C3 |
| MCEMP1 | 102172348 | 6.436 | 7.4E−08 | mast cell expressed |
| | | | | membrane |
| | | | | protein 1 |
| IL34 | 102173115 | 5.434 | 0.0084 | interleukin 34 |
| IL12A | 100861293 | 3.907 | 0.0035 | interleukin 12A |
| TLR4 | 100860955 | 3.423 | 3.8E−07 | toll like receptor 4 |
| TNF | 100861232 | 3.399 | 0.0003 | tumor necrosis factor |
| IL18 | 100861190 | −4.441 | 3.6E−06 | interleukin 18 |
| IL9 | 102179848 | −4.802 | 0.0012 | interleukin 9 |
| IL9R | 102191479 | −4.961 | 9.4E−08 | interleukin 9 receptor |
| IL5 | 102188034 | −4.964 | 0.0396 | interleukin 5 |
| IL36B | 102182235 | −9.557 | 0.0001 | interleukin 36 beta |
| IL13 | 102187477 | −9.675 | 3.4E−07 | interleukin 13 |
| PIWIL2 | 102173845 | −22.152 | 0.0009 | piwi like RNA- |
| | | | | mediated gene |
| | | | | silencing 2 |
| IL11 | 102184367 | −46.823 | 1.6E−07 | interleukin 11 |

Several genes involved in immune responses were significantly regulated in all goat groups. For example, leukemia inhibitory factor (LIF), interferon-gamma (IFN-γ), and interleukin 22 (IL-22), were found to be DE genes in the infected group when compared to both the control and the LAV-vaccinated groups. More gene lists are provided in the Tables included in Appendices A-G. In the infected group, LIF was down-regulated by −2.51 fold change when compared to the control group and by 3.84 fold when compared to the vaccinated group. IL-22, a Th17-related cytokine, was also down-regulated by a −5.78 fold in the infected group vs the control group and by −33.82 fold when compared to the LAV-vaccinated group. Interestingly, NOS2 gene involved in controlling infection of a closely related mycobacteria, M. tuberculosis (Kutsch et al., 1999; Velez et al., 2009), was significantly induced (>100 fold) in both vaccine groups, suggesting an important role of this gene in adaptive immune responses following immunization with LAV (Table 12) or inactivated (Table 13) vaccine. A group of genes with unique diphasic regulatory responses in both LAV and infected goats included immune response genes (e.g. IFN-γ, Granulysin) as well as basic cell metabolic process (e.g. ART5). This list of genes (Table 14) could expand gene categories utilized as targets for developing a sensitive assay to differentiate infected from vaccinated animals (DIVA).

TABLE 14
Common differentially expressed genes regulated in opposite direction
between 30 days post-infection and 30 days post-LAV-vaccinated
groups, each compared to the naïve group

| | | Fold | Fold | |
|---|---|---|---|---|
| | | change in | change in | |
| Gene symbol | Gene ID | Infected group | Vaccinated group | Description |
| LOC106503226 | 106503226 | 2.62 | −2.53 | non-coding RNA |
| PMP22 | 102184371 | 2.11 | −3.46 | peripheral myelin protein 22 |
| ART5 | 102169686 | −2.01 | 3.05 | ADP-ribosyltransferase 5 |
| LOC102169116 | 102169116 | −2.03 | 2.27 | ecto-ADP-ribosyltransferase |
| | | | | 5 |
| GNLY | 102191341 | −2.13 | 2.19 | granulysin |
| ASAP3 | 102182646 | −2.16 | 2.10 | ArfGAP with SH3 domain |
| | | | | ankyrin repeat and PH |
| | | | | domain 3 |
| LOC108633178 | 108633178 | −2.68 | 2.95 | granzyme B-like |
| TBKBP1 | 102172659 | −3.03 | 2.23 | TBK1 binding protein |
| | | | | transcript |
| SLC17A7 | 102169042 | −3.12 | 5.50 | solute carrier family 17 |
| | | | | member 7 |
| LOC108638192 | 108638192 | −3.27 | 5.28 | non-coding RNA |
| IFNG | 100860815 | −3.36 | 3.89 | interferon gamma |

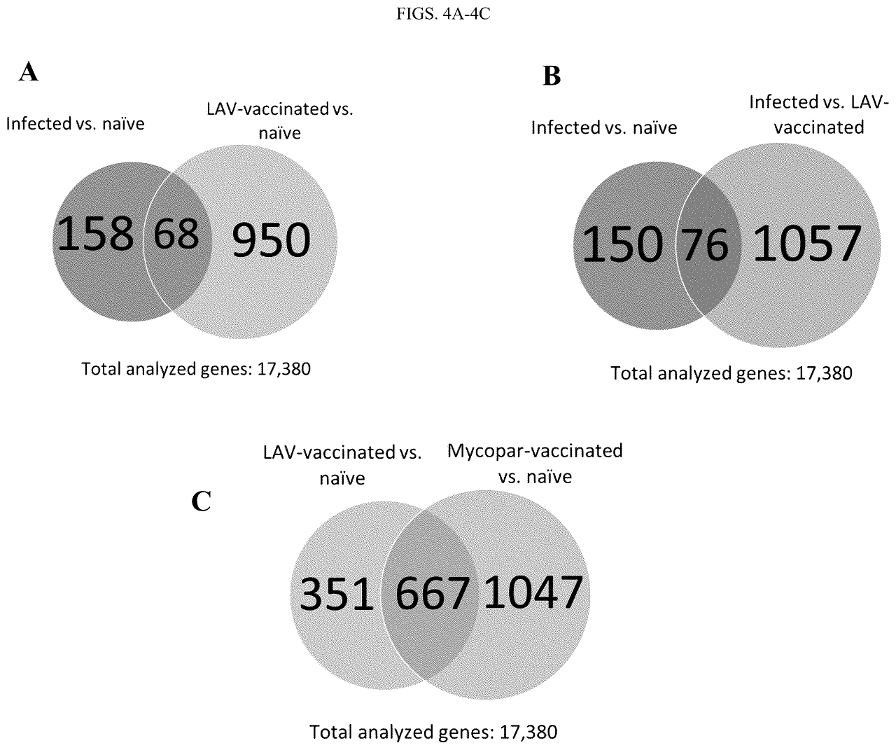

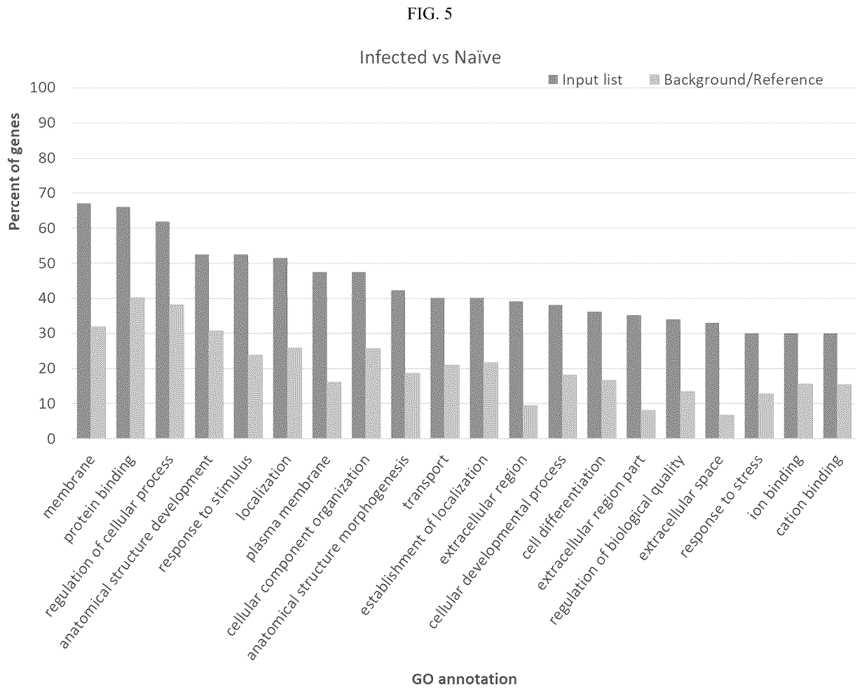

Among those identified DE transcripts in the infected and LAV-vaccinated groups (each referenced against the naïve group), there were 68 transcripts in common (FIG. 4A). The majority of those transcripts were regulated in the same direction in both groups, but 11 transcripts were regulated in the opposite direction. A non-coding RNA transcript, LOC106503226 and a gene, PMP22, were the only two that were up-regulated 30 days PI and down-regulated 30 days post-vaccination. The remaining 9 transcripts (e.g. ART5 and IFNG) were down-regulated 30 days PI and up-regulated 30 days post-vaccination (Table 14). More comparative analysis of transcript profiles identified 76 transcripts commonly up- or down-regulated shared between the lists of genes from comparing infected vs. naïve control and M. paratuberculosis-infected vs. LAV-vaccinated transcripts (FIG. 4B). Those common genes could be considered the core responsive genes for M. paratuberculosis infection or vaccination with an LAV vaccine. For the inactivated vaccine, a total of 667 core genes were also regulated when compared to the LAV-vaccine group (Appendix F). Such core genes included those with potential rules in immunity (e.g. NOS2, RETN and IL21), another indication of core genes responsive to any M. paratuberculosis-specific vaccines whether live-attenuated or inactivated were used.

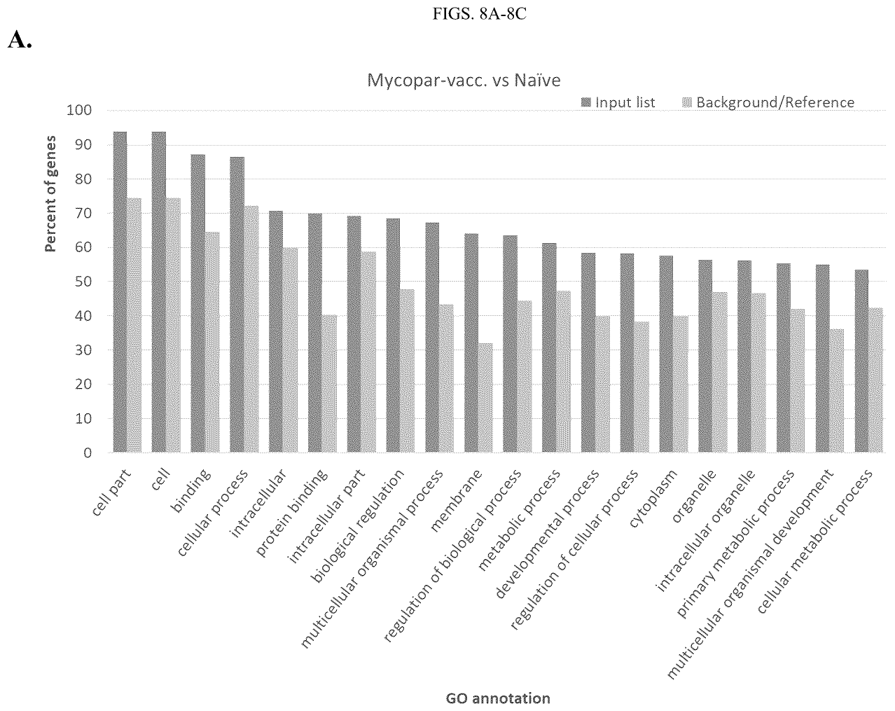

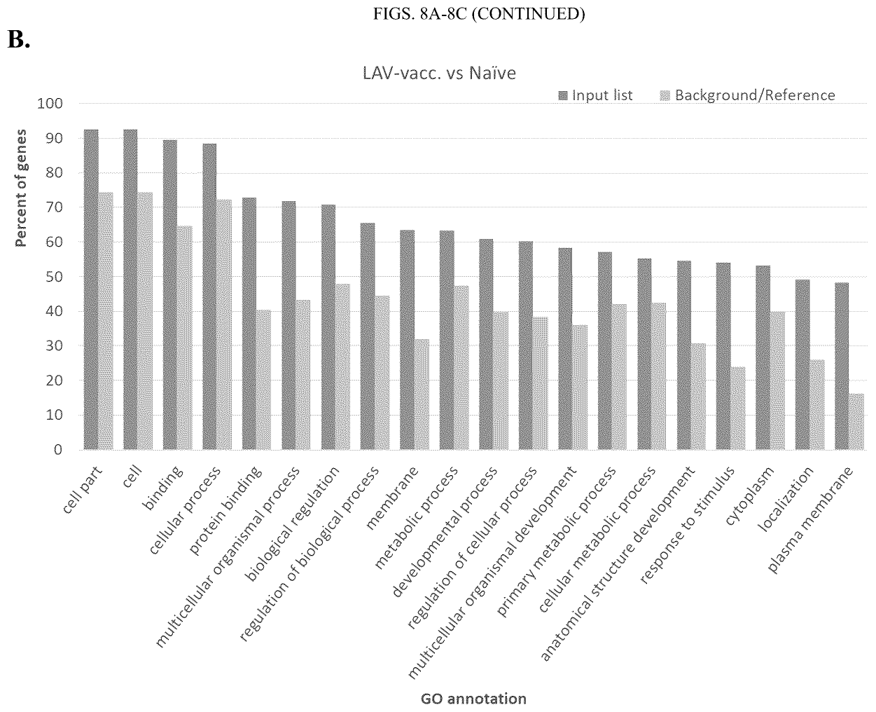

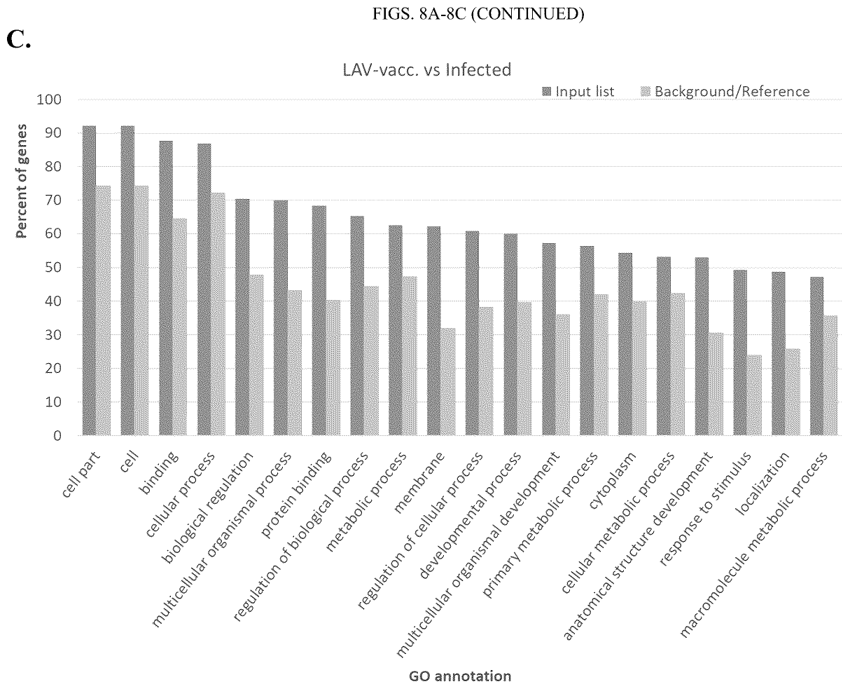

Pathways and networks of differentially expressed genes—To better define gene pathways involved in M. paratuberculosis infection, genes with significant differential expression were evaluated through gene ontology (GO) analysis using agriGO. This analysis provides categories of genes involved in different biological or molecular functions and those integral for different cellular components. Interestingly, the most abundant significant terms for the GO analysis for the infected vs naïve control group included genes involved in protein binding, regulation of cellular process and response to stimulus, which includes significant subcategories immune responses (GO:0006955) and inflammatory response (GO:0006954) (FIG. 3), suggesting the importance of controlling immune genes by M. paratuberculosis following infection. On the other hand, the largest gene groups with significant GO terms for the Mycopar®- or LAV-vaccinated vs infected groups included genes involved in binding, cellular process and metabolic process while those for the LAV-vaccinated vs infected group included genes involved in cellular process and biological regulation (FIGS. 8A-8C).

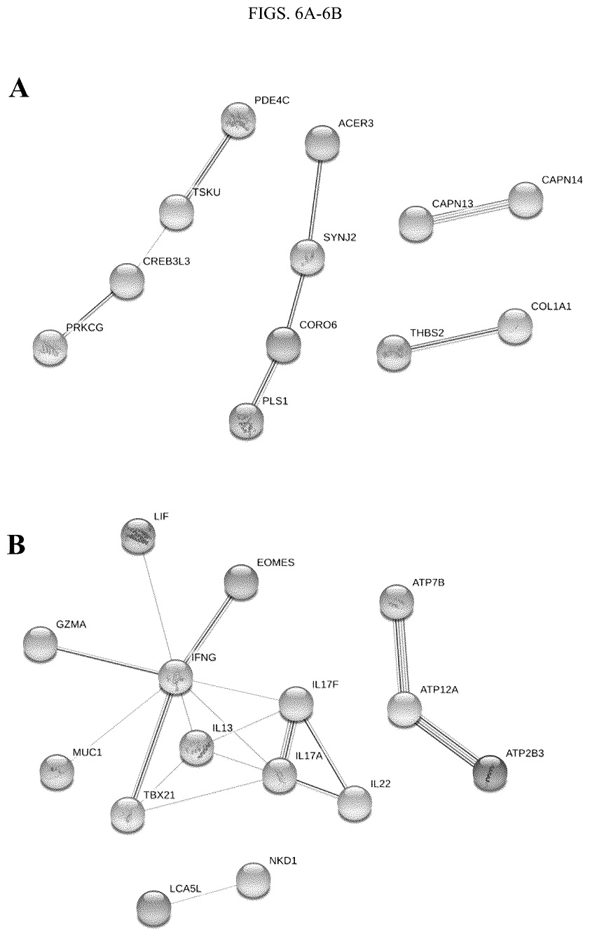

To better characterize gene networks activated during infection and vaccination, gene transcripts were further analyzed to identify co-regulated genes. FIGS. 6A-6B display gene network analysis in the post-infection group. Several in the up-regulated group of genes (FIG. 6A), such as ACER3, SYNJ2, CORO6 and PLS1, showed physical associations and co-expression among transcripts of the M. paratuberculosis-infected group. In addition, homologs of PDE4C and TSKU were also found associated (Halls and Cooper, 2010; Schlecht et al., 2012; Costanzo et al., 2016) and suggested to be involved in signaling and relaxin regulation (Halls and Cooper, 2010). In FIG. 6B, a co-down-regulation of ATP7B, ATP12A and ATP2B3 suggests a possible reduced activity of calcium transport in infected cells. This analysis also highlighted the negative regulation by M. paratuberculosis of host cytokines such as IFN-γ, IL-13, IL-17A, IL-17F and IL-22.

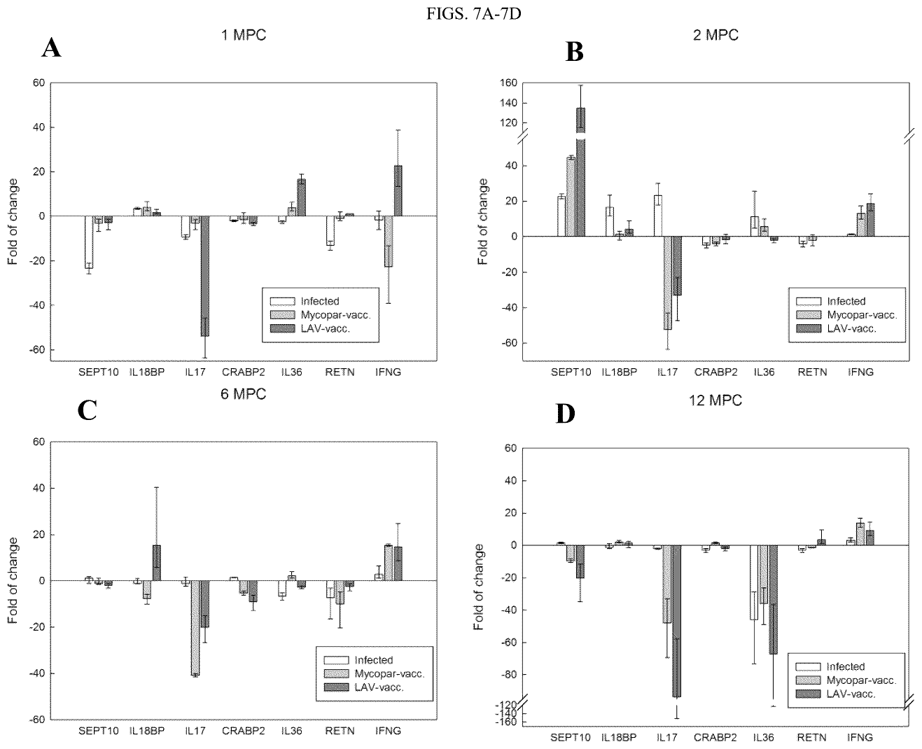

Prolonged changes of key host genes. To further analyze the utility of transcriptome analysis for prediction of unique transcripts associated with infection or vaccination, we used real-time, quantitative PCR to compare transcript levels among animal groups over 12 months post-challenge (MPC) (FIGS. 7A-7D). Interestingly, IL-17 cytokine was repressed in the challenged and Mycopar® and LAV-vaccinated goats compared to the naïve control group for all examined times, except for the infected group at 2 MPC. Similarly, the Sept10 gene was induced, only at 2 MPC. On the other hand, IL-36 was activated soon after vaccination (1 and 2 MPC) but then repressed for the rest of the examined time points, i.e. 6 and 12 MPC. More interestingly, the IFN-γ expression profile was refractive to elicited immune responses. IFN-γ was induced soon in the LAV-vaccine group (1 MPC) but then continued to be expressed in the Mycopar®-vaccinated and M. paratuberculosis-challenged groups starting from 2 MPC until the end of the experiment. At all of these sampling times, the IFN-γ was consistently higher in the LAV-vaccine group compared to the challenged group.

Differential Expression in LAV vaccinated animals, Mycopar™ vaccinated animals, and infected animals.

TABLE 15
List of host genes (goat and cow) differentially expressed in both LAV
and Mycopar vaccinated animals compared to infected animals

| | | | | Mycopar | | | |
|---|---|---|---|---|---|---|---|
| | | LAV vs. Infected | | vs Infected | | Fold | |
| Gene | | Fold | | Fold | | change | |
| symbol | Gene ID | change | p-value | change | p-value | difference | Description |
| STC1 | 102179386 | −4.668 | 1.4E−07 | −8.937 | 5.6E−17 | 4.268 | stanniocalcin 1 |
| NGF | 100862660 | −1.778 | 2.8E−02 | −5.001 | 1.2E−04 | 3.223 | nerve growth |
| | | | | | | | factor |
| FAM150B | 102183516 | −1.406 | 4.8E−02 | −4.355 | 2.0E−05 | 2.949 | family with |
| | | | | | | | sequence |
| | | | | | | | similarity 150 |
| | | | | | | | member B |
| FOXE1 | 106502413 | −1.679 | 1.4E−02 | −4.290 | 4.5E−07 | 2.611 | forkhead box E1 |
| C28H10orf71 | 102181364 | −2.870 | 5.2E−03 | −5.463 | 1.3E−04 | 2.593 | chromosome 28 |
| | | | | | | | C10orf71 |
| | | | | | | | homolog |
| HEBP2 | 102174123 | −1.584 | 4.2E−02 | −4.133 | 1.9E−03 | 2.549 | heme binding |
| | | | | | | | protein 2 |
| IL11 | 102184367 | −2.218 | 8.9E−03 | −4.659 | 1.1E−05 | 2.440 | interleukin 11 |
| KRT82 | 102183763 | −1.630 | 5.6E−03 | −3.951 | 4.0E−04 | 2.320 | keratin 82 |
| NTNG1 | 102190191 | −1.704 | 8.1E−06 | −3.726 | 7.9E−16 | 2.022 | netrin G |
| SORCS2 | 102176511 | −1.357 | 4.9E−03 | −3.365 | 5.8E−09 | 2.008 | sortilin related |
| | | | | | | | VPS10 domain |
| | | | | | | | containing |
| | | | | | | | receptor 2 |
| HS3ST2 | 102183286 | −3.638 | 1.0E−07 | −1.432 | 3.4E−02 | −2.206 | heparan sulfate- |
| | | | | | | | glucosamine 3- |
| | | | | | | | sulfotransferase 2 |
| TGFB3 | 102189962 | 2.706 | 8.5E−03 | 5.103 | 4.2E−06 | −2.397 | transforming |
| | | | | | | | growth factor |
| | | | | | | | beta 3 |

TABLE 16
List of host genes (goat and cow) differentially expressed in LAV
vaccinated animals compared to infected animals but not differentially expressed in Mycopar ®
vaccinated animals compared to infected animals.

| | | LAV | | Mycopar | | |
|---|---|---|---|---|---|---|
| | | vs. Infected | | vs Infected | | |
| Gene | | Fold | | Fold | | |
| symbol | Gene ID | change | p-value | change | p-value | Description |
| LOC102176439 | 102176439 | 3.865 | 0.003 | 0.763 | 0.603 | misc_RNA |
| LOC102187130 | 102187130 | 3.341 | 0.002 | −0.300 | 0.800 | protein ARMCX6-like |
| LOC108633178 | 108633178 | 2.976 | 0.000 | 0.895 | 0.129 | granzyme B-like |
| CPNE6 | 102180500 | 2.755 | 0.000 | 0.841 | 0.289 | copine 6 |
| IL13 | 102187477 | 2.752 | 0.000 | −0.220 | 0.752 | interleukin 13 |
| CCR10 | 102184001 | 2.581 | 0.007 | 0.700 | 0.502 | C—C motif chemokine |
| | | | | | | receptor 10 |
| C1QL2 | 102176742 | 2.427 | 0.032 | −0.520 | 0.684 | complement C1q like 2 |
| MGAT3 | 102185445 | 2.418 | 0.005 | −0.570 | 0.553 | mannosyl (beta-4-)- |
| | | | | | | glycoprotein beta-4-N- |
| | | | | | | acetylglucosaminyltransferase |
| GNLY | 102191341 | 2.212 | 0.000 | 0.807 | 0.146 | granulysin transcript |
| KY | 102169426 | −3.013 | 0.003 | −0.144 | 0.884 | kyphoscoliosis peptidase |
| RAET1E | 108636743 | −3.784 | 0.002 | −0.895 | 0.497 | retinoic acid early transcript |
| | | | | | | 1E |

TABLE 17
List of host genes (goats and cow) differentially expressed in Mycopar ®
vaccinated animals compared to infected animals but not differentially expressed in LAV
vaccinated animals compared to infected animals.

| | | LAV vs. | | Mycopar | | |
|---|---|---|---|---|---|---|
| | | Infected | | vs Infected | | |
| Gene | | Fold | | Fold | | |
| symbol | Gene ID | change | p-value | change | p-value | Description |
| LOC102174895 | 102174895 | 1.762 | 0.141 | 5.278 | 0.000 | vascular cell adhesion protein |
| BMP10 | 102185577 | 0.557 | 0.721 | 4.192 | 0.012 | bone morphogenetic protein |
| | | | | | | 10 |
| CXCL12 | 102169556 | −0.755 | 0.302 | 3.613 | 0.000 | C—X—C motif chemokine |
| | | | | | | ligand 12 |
| LOC108633303 | 108633303 | −0.272 | 0.769 | 2.659 | 0.008 | platelet glycoprotein 4-like |
| PPARG | 100861309 | 0.904 | 0.053 | 2.339 | 0.000 | peroxisome proliferator |
| | | | | | | activated receptor gamma |
| F13A1 | 102169238 | −0.132 | 0.883 | 2.239 | 0.021 | coagulation factor XIII A |
| | | | | | | chain |
| LOC108634012 | 108634012 | −0.173 | 0.804 | −2.736 | 0.000 | homeobox protein MSX-3-like |
| LRRC3 | 102188902 | 0.357 | 0.786 | −2.907 | 0.042 | leucine rich repeat containing 3 |
| MYO10 | 102175716 | −0.967 | 0.023 | −3.183 | 0.000 | myosin X |
| LOC102179419 | 102179419 | −0.471 | 0.759 | −3.435 | 0.040 | myeloid-associated |
| | | | | | | differentiation marker |

### Discussion

Infection with M. paratuberculosis is costing the dairy industry significant economic losses (Cho et al., 2012) and is difficult to detect its presence, especially during early disease stages (Li et al., 2017). In this project, the goat PBMC transcriptome was profiled using RNA-Sequencing (RNA-Seq) to compare the early gene expression, 30 days post-infection and post-vaccination, compared to healthy, naïve controls. In addition to better understanding of disease progression, such analysis is expected to yield targets for further development into a diagnostic assay for early stages of Johne's disease. Many transcriptomic analyzing tools largely depend on information from an annotated genome. In this study, our quality of transcriptomic analyses improved as the goat genome assembly was significantly refined (Bickhart et al., 2017). According to NCBI Capra hircus Annotation Release 102, of 20,593 predicted coding genes, 20,256 had a protein aligned 50% or more of the query against the UniProtKB/Swiss-Prot curated proteins (NCBI, 2016). The updated annotation thus provides a much more reliable reference to our analysis. The generated RNA-Seq dataset could also benefit further improvement of goat genome annotation. As expected, a large number of differentially expressed (DE) transcripts were found between the vaccinated and infected groups (1133 genes) and between the vaccinated and naïve control group (1018 genes). In contrast, there was a relatively small number (226) of DE transcripts when comparing the infected and naïve control group. This large difference in the number of DE transcripts is most likely associated with the route of administration since both vaccines were administered subcutaneously (contrary to oral infection), allowing for increased contact with PBMCs in the bloodstream, while challenge dose of M. paratuberculosis could reach PBMC following intestinal invasion (Stabel et al., 2009). Our analysis, further illustrated the importance of route of infection and/or vaccination for the type and magnitude of the generated host responses.

Although the comparison between the infected and naïve control group produced a relatively small number of DE transcripts, preliminary evaluation of these genes indicated a large number of genes with immunological and inflammatory functions, including interferon gamma (IFN-γ), IL-18 binding protein, IL-17A, and IL-22. IFN-γ is an important player in the defense against intracellular pathogens including mycobacteria (Arsenault et al., 2012). A previous study in cattle showed that in the subclinical stages of infection, IFN-γ expression increased at the site of infection (Sweeney et al., 1998). Other studies indicate that M. paratuberculosis-infected animals produce IFN-γ but are unresponsive to it (Arsenault et al., 2012). In that study, IFN-γ was secreted significantly less (−3.36 fold change) in subclinically infected goats compared with the naïve, control goats. This IFN-γ profile was also evident in subclinically infected goats vs vaccinated goats (−13.0 fold change). Previously, IFN-γ was reported to be induced in PBMC's stimulated with M. paratuberculosis whole-cell sonicate from subclinically infected cows (Stabel, 2000). However, these cows ranged from 2-10 years of age and therefore were much further along in the infection pathogenesis than in the current study, which tested goats 30 days PI. The host response clearly changes over time and this data may demonstrate that. Potentially linked to the identified repression of IFN-γ, is the moderate up-regulation (+1.30 fold change) of interleukin 18 binding protein (IL-18 bp) in the infected vs naïve control group. IL-18 bp binds to IL-18 to block its biological activity (Novick et al., 1999). IL-18 is a pro-inflammatory cytokine that functions in the early Th1 cytokine response and induces IFN-γ production. A major source of IL-18 bp is from intestinal endothelial cells and macrophages (Corbaz et al., 2002). Therefore, IL-18 bp serves to modulate the early Th1 immune response in the intestine, the site of M. paratuberculosis infection. Interestingly, IL-18 bp has been found to be up-regulated during active Crohn's disease, an inflammatory bowel disease in humans with potential association to M. paratuberculosis infection (Corbaz et al., 2002).

As expected, genes involved in immune responses (e.g. LIF, IFN-γ and IL-22), were found to be DE among examined goat groups. LIF is a pleiotropic cytokine belonging to the IL-6 cytokine family with receptors primarily on monocytes/macrophages (Nicola and Babon, 2015). In the infected group, both LIF and IL-22, a Th17-related cytokine, were down-regulated in the infected group vs the control or the vaccinated groups. These three genes, along with IL-13 and IL-17, were also found having associations in the protein network analysis. IL-17 was also down-regulated in the infected vs control group. Down-regulation of IFN-γ, IL-22 and IL-17 genes may suggest overall down-regulation of Th1 and Th17 cell activities and reduced cellular immunity against infections. Several studies in Mycobacterium tuberculosis and Mycobacterium bovis have shown significant IL-17 responses (Blanco et al., 2011; Jurado et al., 2012). A recent study on RNA-Seq analysis in cattle infected with M. bovis showed an up-regulation of IL-17, IL-22, and IFN-γ at one-month PI (Waters et al., 2015). This is in contrast to some of our findings in the present study (in case of IL-17) which was further confirmed by prolonged analysis of key genes up to 12 months post infection (FIGS. 7A-7D). Such difference could be attributed to the difference in host response to M. bovis vs. M. paratuberculosis. Further investigation into these key immune regulated genes as will aid in understanding how the host is dynamically responding to M. paratuberculosis infection or vaccination.

Our gene network analysis also shows associations among genes that were up-regulated in the infected group (FIG. 7A). Interestingly, homologs of ACER3, SYNJ2, CORO6 and PLS1 in animal species other than goats (mainly bovine, Bos Taurus) were also shown to have physical associations (Schlecht et al., 2012; Hein et al., 2015) and co-expression (Clancy et al., 2003; Janji et al., 2010) as well. Particularly, homologs of CORO6, an actin binding protein, was suggested to be involved in cytokinesis. In M. tuberculosis-infected macrophages, CORO6 homolog coronin-1a was suggested to inhibit auto-phagosome formation and facilitate M. tuberculosis survival (Seto et al., 2012). In addition, homologs of PDE4C and TSKU were also found associated (Halls and Cooper, 2010; Schlecht et al., 2012; Costanzo et al., 2016) and suggested to be involved in signaling and relaxin regulation (Halls and Cooper, 2010). It may thus imply a status of progression of an M. paratuberculosis infection in hosts as observed in M. tuberculosis infection (Seto et al., 2012). This observation, along with the likely reduced cellular immunity discussed above, is consistent with the infection status of the host. It is unclear, however, how bacterial or host factors regulate the expression of those genes. Understanding the host-pathogen interaction early in infection will allow for the identification of genes upregulated during initial infection. A useful biomarker for infection must be specific, detectable over the course of the disease with varying inoculation doses, and easily measurable. Moreover, it would improve interpretation of early disease detection if the biomarkers could differentiate infected and vaccinated animals. In our analyses, we identified 9 transcripts (out of 11 in Table 14) that were down-regulated 30 days PI and up-regulated 30 days post-vaccination. This biphasic regulation of those genes or transcripts might make them specific markers for differentiating vaccinated animals that are healthy or those infected with M. paratuberculosis.

The RNA-Seq analysis was performed only on samples taken one month post-infection or post-vaccination to identify early gene regulations in tested groups, notably, between one month after vaccinated only and infected only groups. This comparison differentiates host gene regulating responses after exposure to vaccine strains or virulent strains of M. paratuberculosis. The vaccinated animals were then challenged two months after the vaccination and several key gene expressions were profiled with quantitative PCR (FIGS. 7A-7D). The temporal expression patterns within the tested one year period could reflect unique characteristics of host responses after exposure to virulent M. paratuberculosis with or without prior vaccinations and could also benefit development of diagnostics. For example, IL-17 expressions in the vaccinated animals remained highly repressed at all time while peaking at 2 month post-challenge in the infected only group.

We claim:

1. A method for diagnosis of mycobacterial infection in a mammal, the method comprising the steps of
a) obtaining a first type of sample from the mammal;
b) measuring the expression level in the sample of at least one biomarker selected from the group consisting of LOC108634521, LOC108637251, LOC108637252, LOC108634594, FAM198B, LOC108637671, CDCP1, TMTC1, BAIAP2L1, MEI1, SEPT10, IFNG, IL17F, FCER2, ADGRG1, APBB1, PIWIL2, AOAH, and homologs thereof and comparing the level of the biomarker against the level detected in a first type of sample from an uninfected mammal of the same species; and
c) determining the infection status of the mammal, wherein differential expression of the biomarker is indicative of a mycobacterial infection in the mammal.

2. The method of claim 1, wherein the method is used for early diagnosis and detection of mycobacterial infection in a mammal.

3. The method of claim 1, wherein the measuring is via ELISA assay for antibodies formed against the biomarker.

4. The method of claim 1, wherein measuring is via quantitative PCR.

5. The method of claim 1, wherein the sample is a blood sample.

6. The method of claim 1, wherein the mammal is selected from the group consisting of cow, sheep and goat.

7. A method for differentiating between mammals who have been vaccinated with a live-attenuated mycobacterial vaccine and non-vaccinated mammals, the method comprising the steps of
a) obtaining a first type of sample from the mammal;
b) measuring the expression level in the sample of at least one biomarker selected from the group consisting of LOC108634521, NOS2, LOC108637251, TINAGL1, RETN, C1QL2, TDRD10, TGFB3, ADGRE2, LIPG, KCNJ2, AQP9, BPI, IL9, IL1R2, IL36B, IGF1, BGN, PIWIL2, RAET1E, CRABP2, AOAH and homologs thereof and comparing the level of the biomarker against the level detected in a first type of sample from a non-vaccinated mammal of the same species; and
c) determining the vaccination status of the mammal, wherein differential expression of the biomarker is indicative of a mammal who has been vaccinated with a live-attenuated mycobacterial vaccine.

8. The method of claim 7, wherein the measuring is via ELISA assay for antibodies formed against the biomarker.

9. The method of claim 7, wherein the measuring is via quantitative PCR.

10. The method of claim 7, wherein the sample is a blood sample.

11. The method of claim 7, wherein the mammal is selected from the group consisting of cow, sheep and goat.

12. The method of claim 7, wherein the live-attenuated mycobacterial vaccine is a Mycobacterium mutant vaccine.

13. The method of claim 12, wherein the Mycobacterium mutant vaccine comprises at least one mutation in at least one gene sequence encoding global gene regulators (GGRs) selected from the group consisting of sigH, sigL and LipN.

14. A method for differentiating between mammals who have been vaccinated with a live-attenuated mycobacterial vaccine and infected mammals, the method comprising the steps of
a) obtaining a first type of sample from the mammal;
b) measuring the expression level in the sample of at least one biomarker selected from the group consisting of LOC106503226, PMP22, ART5, LOC102169116, GNLY, ASAP3, LOC108633178, TBKBP1, SLC17A7 and homologs thereof and comparing the level of the biomarker against the level detected in a first type of sample from an infected mammal of the same species; and
c) determining the vaccination status of the mammal, wherein differential expression of the biomarker is indicative of a mammal who has been vaccinated with a live-attenuated mycobacterial vaccine.

15. The method of claim 14, wherein the measuring is via ELISA assay for antibodies formed against the biomarker.

16. The method of claim 14, wherein the measuring is via quantitative PCR.

17. The method of claim 14, wherein the sample is a blood sample.

18. The method of claim 14, wherein the mammal is selected from the group consisting of cow, sheep and goat.

19. The method of claim 14, wherein the live-attenuated mycobacterial vaccine is a Mycobacterium mutant vaccine.

20. The method of claim 14, wherein the Mycobacterium mutant vaccine comprises at least one mutation in at least one gene sequence encoding global gene regulators (GGRs) selected from the group consisting of sigH, sigL and LipN.
